(19) United States
(12) Patent Application Publication
(10) Pub. No.: US 2024/0052417 A1
(43) Pub. Date: Feb. 15, 2024

(54) METHOD FOR EARLY DETERMINATION OF GENDER BY MULTIPLEX PCR DETECTING FOUR GENES

(71) Applicants: Imam Abdulrahman Bin Faisal University, Dammam (SA); King Saud University, Riyadh (SA)

(72) Inventors: J. Francis BORGIO, Dammam (SA); Norah Fahad ALHUR, Riyadh (SA); Nourah H. AL QAHTANI, Dammam (SA); Sayed ABDULAZEEZ, Dammam (SA); Noor B. ALMANDIL, Dammam (SA); Entissar Asulaiman ALSUHAIBANI, Riyadh (SA)

(73) Assignees: Imam Abdulrahman Bin Faisal University, Dammam (SA); King Saud University, Riyadh (SA)

(21) Appl. No.: 17/819,472

(22) Filed: Aug. 12, 2022

(51) Int. Cl. C12Q 1/6883; C12Q 1/6806

(52) U.S. Cl. CPC C12Q 1/6883; C12Q 1/6806; C12Q 2600/156

(57) ABSTRACT

A non-invasive method for determining fetal gender prior to the second trimester using multiplex polymerase chain reaction to identify target segments of the SRY, DAZ2 and TSPY1 genes which are located on the Y chromosome of a male fetus. A kit for determining gender comprising primers that amplify target segments of the SRY, DAZ2 and TSPY1 genes.

### REFERENCE TO A SEQUENCE LISTING

In accordance with 37 CFR § 1.52(e)(5), the present specification makes reference to a Sequence Listing submitted electronically as a .xml file named “537239US_ST26.xml”. The .xml file was generated on Aug. 10, 2022 and is 4,633 bytes in size. The entire contents of the Sequence Listing are hereby incorporated by reference.

### BACKGROUND OF THE INVENTION

### Field of the Invention

The invention falls within the fields of medicine including those of obstetrics and gynecology and molecular genetics. It pertains to non-invasive methods for determining the gender of a fetus early in a pregnancy using a multiplex polymerase chain reaction that amplifies three different Y chromosome markers: SRY, DAZ2 AND TSPY1 and one control gene marker ACTB.

### Description of the Related Art

Fetal gender determination was first developed for prevention or management of X-linked inherited diseases. Over a hundred X-linked inherited diseases have been discovered in humans. These include muscular dystrophy, fragile X syndrome, and hemophilia. X-linked recessive diseases most often occur in males because have only one X chromosome and a single recessive gene on that X chromosome will cause the disease.

When a mother carries an X-linked disease genotype, where she typically carries only one abnormal X chromosome and the father is not a carrier for the disease, the expected outcome of the pregnancy is: 25% chance of a healthy boy; 25% chance of a boy with disease; 25% chance of a healthy girl; and 25% chance of a girl who carries the disease genotype. If the father has the disease and the mother is not a carrier, the expected outcomes are: 50% chance of a having a healthy boy; or 50% chance of a having a girl without the disease who is a carrier. This means that none of the children would show the signs of the disease, but the trait could be passed to grandchildren. Thus, determination of gender is important for assessing outcome of a pregnancy.

Sonography is conventionally used to determine gender; however, ultrasound can only be conducted in the second trimester when external genitalia are fully developed. In contrast, gender determination using fetal DNA can be performed earlier in pregnancy and is more reliable as it detects genetic markers of gender and does not depend on interpretation of sonographic images.

The source of fetal DNA is often crucial for obtaining a detectable amount or concentration of DNA. Fetal DNA can be obtained by invasive procedures such as chronic villus and amniocentesis sampling or by non-invasive methods, typically from maternal fluids, such as blood or urine. Such fluids can be used as sources of cell free fetal DNA (cffDNA) which enters the maternal circulation from the fetus and placenta. Unfortunately, most non-invasively obtained samples contain only low amounts or concentrations of cell-free fetal DNA (cffDNA).

For male gender determination, the presence of the Y chromosome is detected in non-invasively obtained maternal samples, because the maternal DNA complement does not contain the Y chromosome. Detection of the Y chromosome distinguishes between DNA from a male fetus and DNA from a maternal sample, such as blood, plasma, serum or urine obtained from a pregnant woman.

Various Y chromosome sequences have been used for gender determination. For example, the Y chromosome contains a “male-determining gene,” known as the SRY gene. This gene causes testes to form in the embryo and results in development of external and internal male genitalia. However, methods for detecting the single copy SRY gene are not sensitive enough to accurately determine gender.

Mokari-Zadeh, N., et al, J. CLIN. DIAGNOS. RES. 2015 July, Vol-9(7): GC01-GC04, describes an attempt to develop a Real-Time duplex PCR for determination of fetal gender by gender using two pairs of primers that each recognize the single-copy SRY sequence in ffDNA (free fetal DNA in maternal serum) taken at 8-12 weeks gestation. It describes normal, non-multiplex PCR but does not describe multiplex PCR using primers which identify different Y chromosome targets, not just the single-copy SRY gene.

Hamid Reza Khorram Khorshid, et al., ACTA MEDICA IRANICA, 2013; 51(4): 209-214, identifies individual SRY, DYS14 & DAZ sequences which are not present in the maternal genome using normal, non-multiplex Real-Time PCR. It does not mention multiplex PCR.

F. Javier Fernández-Martinez, et al. describes fetal sex determination in maternal plasma using a specific protocol for cffDNA extraction combined with the application of TaqMan MGB probes for the detection of Y-chromosome single-copy and multicopy sequences following a stringent algorithm. Taqman MGB probes are used for detection of Y chromosome sequences. All regions of the Y chromosome were analyzed in separate reactions and multiplex PCR methods are not disclosed. These methods involve the amplification of single DNA targets and are not multiplex methods which simultaneously amplify multiple target DNAs.

Regrettably, multiplex PCR has a number of problems which complicate its use. One problem is the generation of false negative results such as identifying a male fetus as a female fetus. False negatives in multiplex PCR can be produced when primer binding to a target is inhibited. Other causes include false amplification due to primer dimers, false amplicons, primer-amplicon interactions, and unimolecular extension which are associated with polymerase extension and depletion of primers and dNTPs. Primer binding can be inhibited by formation of secondary structures including hairpins in a target DNA (such as portions of Y chromosome DNA) which compete with a primer for binding to a target sequence.

A reciprocal problem is the production of false positives by multiplex PCR for example, where a female fetus is identified as male. False positives are the result of false hybridization occurring during the PCR.

For multiplex PCR, designing primers to be specific is absolutely critical, because in a multiplex PCR there are so many primers and so many amplicons that the possibility of getting cross-hybridization becomes huge and grows very quickly.

A multiplex PCR is a complex interacting system involving other maternal and fetal DNA besides the target Y chromosome DNA. False amplicons arising from contaminating DNA can involve rare hybridization reactions and weak hybridization reactions, not perfect matches, but hybridizations that involve mismatches or even contain bulges formed of mismatched DNA segments.

Primer designing tools like BLAST which searches based on sequence similarity without additional screening and selection, do not reliably identify suitable multiplex primers. A reason for this is that sequence similarity determined by BLAST is not the same thing as thermodynamic stability between a primer and its target. Thus, BLAST primer design programs without careful supervision or testing cannot be relied upon to predictably solve the problems mentioned above.

In view of the drawbacks of PCR-based technologies for gender determination, the inventors sought to develop a more highly sensitive PCR-based test that could detect male fetal DNA even in sources such as cffDNA containing very low amounts or concentrations of fetal DNA.

As disclosed herein, the inventors have developed a multiplex PCR method that amplifies select Y chromosome DNA sequences in cffDNA using a set of carefully designed, selected, and tested primers. This method provides a convenient, non-invasive, and highly sensitive way to detect Y chromosome sequences in cffDNA comprising a mixture of maternal and fetal DNA, thus determining gender of a fetus even in early stages of pregnancy.

Primers were designed manually and PrimerBlast was used to confirm the annealing sites and ensure the nonspecific annealing for the primers. Manual techniques were used for the adjusting the annealing location and specific melting temperature of the primers. Manual methodologies were adopted to attain and refine specific, selected melting temperatures of the primers.

Permutation combinations of primers were checked for the non-specific amplification for genomic DNA and were checked using multiple sequence alignments and PCR amplifications in the laboratory. Multiple sequence alignments were used for reducing the primer dimer with permutation combinations of primers. Primers were selected so as to not interfere with amplicon size; the smallest amplification was 269 bp which is significantly longer than dimers of about 50 bp.

### BRIEF DESCRIPTION OF THE INVENTION

One aspect of the invention is a non-invasive multiplex PCR-based method for simultaneous detection of three distinct Y chromosome-specific target DNAs and a control target DNA in a biological sample containing fetal DNA, comprising (a) simultaneously amplifying portions of the SRY, DAZ2, TSPY1 and ACTB genes to produce amplicons which each have a different distinguishable length; (b) detecting the presence or absence of amplicons from the SRY, DAZ2 and TSPY1 and ACTB by based on differential lengths of each amplicon.

Male gender is determined when amplicons for at least two of the Y chromosome-specific SRY, DAZ2 or TSPY1 are detected, or when presence of one or more amplicons from SRY, DAZ2 or TSPY1 having an intensity of ≥1,000,000 is detected. Similarly, non-male gender (female gender) is determined when no amplicons from at least two of SRY, DAZ2 or TSPY1 are detected or when only a presence of a single amplicon from SRY, DAZ2 or TSPY1 having an intensity lower than 1,000,000 is detected.

Preferably, the multiplex amplification is performed using a first primer comprising SEQ ID NO: 1 and a second primer comprising SEQ ID NO: 2 which amplify the SRY target nucleic acid sequence; a third primer comprising SEQ ID NO: 7 and a fourth primer comprising SEQ ID NO: 8 which amplify the DAZ2 target nucleic acid sequence; a fifth primer comprising SEQ ID NO: 5 and a sixth primer comprising SEQ ID NO: 6 which amplify the TSPY1 target nucleic acid sequence; and a seventh primer comprising SEQ ID NO: 3 and an eighth primer consisting of SEQ ID NO: 4 which amplify the ACTB target nucleic acid sequence. Optionally, at least one of said primers may comprise one or more modified nucleotides selected from the group consisting of 2′-O-methyl nucleotides, 2′-fluoro-nucleotides, 2′-amino nucleotides, and arabinose nucleotides.

Another aspect of the invention is directed to a kit for determining gender of a subject comprising a set of primers that amplify segments of SRY, DAZ2, TSPY1 and ACTB genes to produce amplicons of different lengths. Preferably, the kit comprises a first primer comprising SEQ ID NO: 1 and a second primer comprising SEQ ID NO: 2 which amplify the SRY target nucleic acid sequence; a third primer comprising SEQ ID NO: 7 and a fourth primer comprising SEQ ID NO: 8 which amplify the DAZ2 target nucleic acid sequence; a fifth primer comprising SEQ ID NO: 5 and a sixth primer comprising SEQ ID NO: 6 which amplify the TSPY1 target nucleic acid sequence; and a seventh primer comprising SEQ ID NO: 3 and an eighth primer consisting of SEQ ID NO: 4 which amplify the ACTB target nucleic acid sequence; and optionally, wherein one or more of said primers comprise one or more modified nucleotides selected from the group consisting of 2′-O-methyl nucleotides, 2′-fluoro-nucleotides, 2′-amino nucleotides, and arabinose nucleotides. Such a kit preferably contains other equipment or supplies for isolating fetal DNA, for performance of multiplex PCR, or for detection of amplicons produced by multiplex PCR.

### BRIEF DESCRIPTION OF THE DRAWINGS

A more complete appreciation of the invention and many of the attendant advantages thereof will be readily obtained as the same becomes better understood by reference to the following detailed description when considered in connection with the accompanying drawings, wherein:

FIG. 1 provides a flow chart describing preferred sources of biological samples containing DNA and some methods for isolation or extraction of DNA from samples for gender determination. Abbreviations: Peripheral blood mononuclear cell (PBMC); cell-free fetal DNA (cffDNA), gDNA; fetal nucleated red blood cell (fNRBC); genomic DNA (gDNA); polymerase chain reaction (PCR).

FIG. 2A shows PCR products produced by amplification of a single marker from a male DNA sample as resolved on an agarose gel. Single marker amplifications of SRY, DAZ2 and TSPY1 are shown.

FIG. 2B shows PCR products produced by amplification of ACTB from a female DNA sample as resolved on an agarose gel.

FIG. 2C shows multiplex PCR results when amplification is performed at different temperatures (temperature gradient).

FIG. 3A shows SRY, ACTB, TSPY1 and DAZ2 amplicons produced by multiplex PCR using buccal cell DNA.

FIG. 3B shows SRY, ACTB, TSPY1 and DAZ2 amplicons produced by multiplex PCR of DNA obtained from blood. Results from male DNA samples are shown in left lanes; results from female DNA samples are shown in the right lanes.

FIG. 4A shows multiplex PCR results after amplification of SRY, ACTB, TSPY1 and DAZ2 using different amounts of male target DNA from blood. Bands from Y chromosome markers are detectable at the lowest concentration tested 0.5 ng.

FIG. 4B shows multiplex PCR results after amplification of ACTB using different amounts of female target DNA from blood. Bands from ACTB are detectable at the lowest concentration tested 0.5 ng.

FIG. 5A shows multiplex PCR results after amplification of SRY, ACTB, TSPY1 and DAZ2 using different amounts of male target DNA from blood. Bands from Y chromosome markers are detectable at the lowest concentration tested 0.2 ng.

FIG. 5B shows multiplex PCR results after amplification of ACTB using different amounts of female target DNA from blood. Bands from ACTB are detectable at the lowest amount tested 0.2 ng.

FIG. 6A shows results of the amplification of SRY, ACTB, TSPY1 and DAZ2 in various samples of target DNA:

ladder/markers (L);

Paternal DNA (F2);

Maternal DNA (M3);

Paternal DNA (PDY2);

Maternal DNA (PDY6);

Maternal urine cffDNA (U10);

Maternal urine cffDNA (U11);

Maternal serum cffDNA (S10);

Maternal serum cffDNA (S12);

Ladder/markers (L);

cffDNA from maternal plasma (MP10);

fetal DNA from CD71⁺ cells isolated from cord blood (M5+);

fetal DNA from CD71⁺ cells isolated from cord blood (C13H);

Fetal DNA isolated from cord blood fetal cells (C59);

Paternal buccal DNA (PDY3);

Maternal buccal cell DNA (PDY8);

Ladder/markers (L).

FIG. 6B shows the results of the amplification of SRY, ACTB, TSPY1 and DAZ2 in various samples of target DNA:

Ladder/markers (L);

Paternal DNA from blood (F4);

Maternal DNA from blood (M4);

fetal DNA from fetal blood (UKN3);

fetal DNA from fetal blood (UNK4);

DNA from unknown tissue (UNK 4 tissue);

fetal DNA of fetal cell isolated from maternal blood (M59).

FIG. 7 illustrates the intensity of amplicons of single Y marker. Red boxes (ten boxes at left) indicate the acceptable intensities. Blue box (last box on right) indicates a less than acceptable intensity.

FIGS. 8A, 8C, 8E, and 8G depict electropherograms of gel-eluted amplicons of multiplex PCR. Electropherograms are generated from the forward primers of the multiplex PCR amplicons. The sequence at the top of FIG. 8A is described by nucleotides 287-337 of SEQ ID NO: 9. The sequence at the top of FIG. 8C is described by nucleotides 381-430 of SEQ ID NO: 12. The sequence at the top of FIG. 8E is described by nucleotides 86-138 of SEQ ID NO: 10. The sequence at the top of FIG. 8G is described by nucleotides 80-128 of SEQ ID NO: 11.

FIGS. 8B, 8D, 8F and 8H depict DNA sequences corresponding to sequences of the amplicons and subsequences shown in the electropherograms of FIGS. 8A, 8C, 8E and 8G, respectively. FIG. 8B (SEQ ID NO: 9) corresponds to the 769 bp amplicon SRY of FIG. 3B. FIG. 8D (SEQ ID NO: 12) corresponds to the 541 bp amplicon ACTB of FIG. 3B. FIG. 8F (SEQ ID NO: 10) corresponds to the 329-356 bp amplicon TSPY1 of FIG. 3B. FIG. 8H (SEQ ID NO: 11) corresponds to the 269 bp amplicon DAZ2 of FIG. 3B. The bolded subsequences correspond to those at the tops of the electrophoretograms of FIGS. 8A, 8C, 8E, and 8G.

### DETAILED DESCRIPTION OF THE INVENTION

As discussed above, there is a need for a simple, accurate, and sensitive method for determining the gender of a fetus early in the term of a pregnancy. With this objective in mind, the inventors sought to identify and evaluate a combination of Y chromosome markers that would make this possible. Rather than relying on detection of a single marker, the inventors sought to develop an internally controlled multiplex PCR method for determining gender of a fetus when only small amounts of fetal DNA were available.

Samples and Samples Preparation Provided herein are methods and compositions for analyzing nucleic acid in a sample. In some embodiments, nucleic acid fragments in a mixture of nucleic acid fragments are analyzed. A mixture of nucleic acids can comprise two or more nucleic acid fragment species having different nucleotide sequences, different fragment lengths, different origins (e.g., genomic origins, fetal and maternal origins, cellular or tissue origins, and the like), or combinations thereof.

Nucleic acid or a nucleic acid mixture utilized in methods, systems, machines and/or apparatuses described herein is often isolated from a sample obtained from a subject. A subject from which a specimen or sample is obtained is sometimes referred to herein as a test subject or patient. A subject may be a male or female (e.g., a woman, a pregnant woman, a pregnant female, or male or female parent). A subject may be any age (e.g., an embryo, a fetus, infant, child, or adult). In some embodiments, a sample may be obtained ex vivo or in vitro, for example, from a forensic sample, from a tissue bank, or from tissue or cell culture.

Nucleic acid may be isolated from any type of suitable biological specimen or sample (e.g., a test sample). A sample or test sample can be any specimen that is isolated or obtained from a subject or part thereof (e.g., a human subject, a pregnant female, a fetus). A test sample is often obtained from a test subject. A test sample is often obtained from a pregnant female (e.g., a pregnant human female).

Non-limiting examples of specimens include fluid or tissue from a subject, including, without limitation, blood or a blood product (e.g., serum, plasma, or the like), umbilical cord blood, chorionic villi, amniotic fluid, cerebrospinal fluid, spinal fluid, lavage fluid (e.g., bronchoalveolar, gastric, peritoneal, ductal, ear, arthroscopic), biopsy sample (e.g., from pre-implantation embryo), celocentesis sample, cells (e.g., blood cells, placental cells, embryo or fetal cells, fetal nucleated cells or fetal cellular remnants) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, the like or combinations thereof.

A test sample often comprises plasma or serum obtained from a pregnant female. In some embodiments, a biological sample is a cervical swab from a subject. In some embodiments, a biological sample may be blood and sometimes plasma or serum. The term “blood” as used herein refers to a blood sample or preparation from a subject (e.g., a test subject such as a pregnant woman or a woman being tested for possible pregnancy).

The term encompasses whole blood, a blood product or any fraction of blood, such as plasma, serum, white blood cells, buffy coat, or the like as conventionally defined. Blood or fractions thereof often comprise nucleosomes (e.g., maternal and/or fetal nucleosomes). Nucleosomes comprise nucleic acids and are sometimes cell-free or intracellular. Blood also comprises buffy coats. Buffy coats can be isolated by utilizing a ficoll gradient. Buffy coats can comprise white blood cells (e.g., leukocytes, T-cells, B-cells, platelets, and the like). In certain embodiments buffy coats comprise maternal and/or fetal nucleic acids.

Blood plasma refers to the liquid fraction of whole blood, for example, a portion resulting from centrifugation of blood treated with anticoagulants.

Blood serum refers to the watery portion of fluid remaining after a blood sample has coagulated, for example, blood plasma without clotting factors or from which the clotting factors have been removed.

Fluid or tissue samples are typically collected in accordance with standard hospital or clinical protocols. For blood, an appropriate amount of peripheral blood (e.g., between 3-40 milliliters) often is collected and can be stored according to standard procedures prior to or after preparation.

A fluid sample from which nucleic acid is extracted may be acellular (e.g., cell-free).

In some embodiments, a fluid or tissue sample may contain cellular elements or cellular remnants. In some embodiments fetal cells or maternal cells may be included in the sample.

A sample often is heterogeneous, by which is meant that more than one type of nucleic acid species is present in the sample. For example, heterogeneous nucleic acid can include, but is not limited to fetal derived and maternal derived nucleic acids. A sample may be heterogeneous because more than one cell type is present, such as a fetal cell and a maternal cell, a cancer and non-cancer cell, or a pathogenic and host cell. In some embodiments, a minority nucleic acid species and a majority nucleic acid species is present.

For prenatal applications of technology described herein, fluid or tissue samples may be collected from a female at a gestational age suitable for testing, or from a female who is being tested for possible pregnancy. Suitable gestational age may vary depending on the prenatal test being performed. In certain embodiments, a pregnant female subject sometimes is in the first trimester of pregnancy, at times in the second trimester of pregnancy, or sometimes in the third trimester of pregnancy. In certain embodiments, a fluid or tissue is collected from a pregnant female between about 1 to about 45 weeks of fetal gestation (e.g., at 1-4, 4-8, 8-12, 12-16, 16-20, 20-24, 24-28, 28-32, 32-36, 36-40 or 40-44 weeks of fetal gestation), and sometimes between about 5 to about 28 weeks of fetal gestation (e.g., at 6, 7, 8, 9, 10, 11, 12, 13, 14, 15, 16, 17, 18, 19, 20, 21, 22, 23, 24, 25, 26 or 27 weeks of fetal gestation). In certain alternative embodiments a fluid or tissue sample is collected from a pregnant female during or just after (e.g., 0 to 72 hours after) giving birth (e.g., vaginal or non-vaginal birth, such as surgical delivery).

Acquisition of Blood Samples and Extraction of DNA. Methods herein often include separating, enriching and analyzing fetal DNA found in maternal blood as a non-invasive means to detect the presence or absence of a maternal and/or fetal genetic variation and/or to monitor the health of a fetus and/or a pregnant female during and sometimes after pregnancy. Thus, the first steps of practicing certain methods herein often include obtaining a blood sample from a pregnant woman and extracting DNA from a sample.

Acquisition of Blood Samples. A blood sample can be obtained from a pregnant woman at a gestational age suitable for testing using a method of the present technology. A suitable gestational age may vary depending on the disorder tested, as discussed below. Collection of blood from a woman is typically performed in accordance with the standard protocols hospitals or clinics generally follow. An appropriate amount of peripheral blood, e.g., typically between 5-50 ml, often is collected and may be stored according to standard procedure prior to further preparation. Blood samples may be collected, stored or transported in a manner that minimizes degradation or the quality of nucleic acid present in the sample.

Preparation of Blood Samples. An analysis of fetal DNA found in maternal blood may be performed using whole blood, serum, or plasma or other suitable samples. Methods for preparing serum or plasma from maternal blood are known. For example, a pregnant woman's blood can be placed in a tube containing EDTA or a specialized commercial product such as Vacutainer SST (Becton Dickinson, Franklin Lakes, N.J.) to prevent blood clotting, and plasma can then be obtained from whole blood through centrifugation. Serum may be obtained with or without centrifugation-following blood clotting. If centrifugation is used then it is typically, though not exclusively, conducted at an appropriate speed, e.g., 1,500-3,000 times g. Plasma or serum may be subjected to additional centrifugation steps before being transferred to a fresh tube for DNA extraction.

In addition to the acellular portion of the whole blood, DNA may also be recovered from the cellular fraction, enriched in the buffy coat portion, which can be obtained following centrifugation of a whole blood sample from the woman and removal of the plasma. In one embodiment, fetal cells, such as trophoblastic (epithelial) cells, are separated from smaller peripheral blood leukocytes.

In some embodiments, the sample may first be enriched or relatively enriched for fetal nucleic acid by one or more methods prior to isolation of nucleic acids. For example, the discrimination of fetal and maternal DNA can be performed using the compositions and processes of the present technology alone or in combination with other discriminating factors. Examples of these factors include, but are not limited to, single nucleotide differences between chromosome X and Y, chromosome Y-specific sequences, polymorphisms located elsewhere in the genome, size differences between fetal and maternal DNA and differences in methylation pattern between maternal and fetal tissues. In certain embodiments, maternal nucleic acid is selectively removed (either partially, substantially, almost completely or completely) from the sample.

The terms “nucleic acid” and “nucleic acid molecule” may be used interchangeably throughout the disclosure. Deoxyribonucleotides include deoxyadenosine, deoxycytidine, deoxyguanosine and deoxythymidine. For RNA, the base thymine is replaced by uracil. These terms refer to nucleic acids of any composition from, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, RNA highly expressed by the fetus or placenta, and the like), and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid may be, or may be from, a plasmid, phage, autonomously replicating sequence (ARS), centromere, artificial chromosome, chromosome, or other nucleic acid able to replicate or be replicated in vitro or in a host cell, a cell, a cell nucleus or cytoplasm of a cell in certain embodiments. A template nucleic acid in some embodiments can be from a single chromosome (e.g., a nucleic acid sample may be from one chromosome of a sample obtained from a diploid organism). Unless specifically limited, the term encompasses nucleic acids containing known analogs of natural nucleotides that have similar binding properties as a reference nucleic acid such as the primers disclosed herein. The term nucleic acid is used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame) and double-stranded polynucleotides.

The term “gene” means the segment of DNA involved in producing a polypeptide chain; it includes regions preceding and following the coding region (leader and trailer) involved in the transcription/translation of the gene product and the regulation of the transcription/translation, as well as intervening sequences (introns) between individual coding segments (exons).

Nucleic Acid Isolation and Processing. Nucleic acid may be derived from one or more sources (e.g., cells, serum, plasma, buffy coat, lymphatic fluid, skin, soil, and the like) by methods known in the art. Nucleic acids are often isolated from a test sample. Any suitable method can be used for isolating, extracting and/or purifying DNA from a biological sample (e.g., from blood or a blood product), non-limiting examples of which include methods of DNA preparation (e.g., described by Sambrook and Russell, MOLECULAR CLONING: A LABORATORY MANUAL 3d ed., 2001), various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), and GFX™ Genomic Blood DNA Purification Kit (Amersham, Piscataway, N.J.), the like or combinations thereof.

Cell lysis procedures and reagents are known in the art and may generally be performed by chemical (e.g., detergent, hypotonic solutions, enzymatic procedures, and the like, or combination thereof), physical (e.g., French press, sonication, and the like), or electrolytic lysis methods. Any suitable lysis procedure can be utilized. For example, chemical methods generally employ lysing agents to disrupt cells and extract the nucleic acids from the cells, followed by treatment with chaotropic salts. Physical methods such as freeze/thaw followed by grinding, the use of cell presses and the like also are useful. High salt lysis procedures also are commonly used. For example, an alkaline lysis procedure may be utilized. The latter procedure traditionally incorporates the use of phenol-chloroform solutions, and an alternative phenol-chloroform-free procedure involving three solutions can be utilized. In the latter procedures, one solution can contain 15 mM Tris, pH 8.0; 10 mM EDTA and 100 μg/ml Rnase A; a second solution can contain 0.2N NaOH and 1% SDS; and a third solution can contain 3M KOAc, pH 5.5. These procedures can be found in CURRENT PROTOCOLS IN MOLECULAR BIOLOGY, John Wiley & Sons, N.Y., 6.3.1-6.3.6 (1989), incorporated herein in its entirety.

Nucleic acid may be isolated at a different time point as compared to another nucleic acid, where each of the samples is from the same or a different source. A nucleic acid may be from a nucleic acid library, such as a cDNA or RNA library, for example. A nucleic acid may be a result of nucleic acid purification or isolation and/or amplification of nucleic acid molecules from the sample. Nucleic acid provided for processes described herein may contain nucleic acid from one sample or from two or more samples (e.g., from 1 or more, 2 or more, 3 or more, 4 or more, 5 or more, 6 or more, 7 or more, 8 or more, 9 or more, 10 or more, 11 or more, 12 or more, 13 or more, 14 or more, 15 or more, 16 or more, 17 or more, 18 or more, 19 or more, or 20 or more samples).

Nucleic acids can include extracellular nucleic acid in certain embodiments. The term “extracellular nucleic acid” as used herein can refer to nucleic acid isolated from a source having substantially no cells and also is referred to as “cell-free” nucleic acid and/or “cell-free circulating” nucleic acid. Extracellular nucleic acid can be present in and obtained from blood (e.g., from the blood of a pregnant female). Extracellular nucleic acid often includes no detectable cells and may contain cellular elements or cellular remnants. Non-limiting examples of acellular sources for extracellular nucleic acid are blood, blood plasma, blood serum and urine. As used herein, the term “obtain cell-free circulating sample nucleic acid” includes obtaining a sample directly (e.g., collecting a sample, e.g., a test sample) or obtaining a sample from another who has collected a sample. Without being limited by theory, extracellular nucleic acid may be a product of cell apoptosis and cell breakdown, which provides basis for extracellular nucleic acid often having a series of lengths across a spectrum (e.g., a “ladder”).

Extracellular nucleic acid can include different nucleic acid species, and therefore is referred to herein as “heterogeneous” in certain embodiments. For example, blood serum or plasma from a person having cancer can include nucleic acid from cancer cells and nucleic acid from non-cancer cells. In another example, blood serum or plasma from a pregnant female can include maternal nucleic acid and fetal nucleic acid.

In some instances, fetal nucleic acid sometimes is about 0.5% to about 50% of the total nucleic acid (e.g., about <0.5, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or 49% of the total nucleic acid is fetal nucleic acid). In some embodiments, the majority of fetal nucleic acid in nucleic acid is of a length of about 500 base pairs or less, about 250 base pairs or less, about 200 base pairs or less, about 150 base pairs or less, about 100 base pairs or less, about 50 base pairs or less or about 25 base pairs or less.

Nucleic acid may be provided for conducting methods described herein without processing of the sample(s) containing the nucleic acid, in certain embodiments. In some embodiments, nucleic acid is provided for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be provided with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components.

The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid provided that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species. For example, fetal nucleic acid can be purified from a mixture comprising maternal and fetal nucleic acid. In certain examples, nucleosomes comprising small fragments of fetal nucleic acid can be purified from a mixture of larger nucleosome complexes comprising larger fragments of maternal nucleic acid.

In some embodiments nucleic acids are fragmented or cleaved prior to, during or after a method described herein. Fragmented or cleaved nucleic acid may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000 or 9000 base pairs. Fragments can be generated by a suitable method known in the art, and the average, mean or nominal length of nucleic acid fragments can be controlled by selecting an appropriate fragment-generating procedure. Typically fragments of a size suitable for amplification using the primers disclosed herein are selected.

The term “amplified” as used herein refers to subjecting a target nucleic acid in a sample to a process that linearly or exponentially generates amplicon nucleic acids having the same or substantially the same nucleotide sequence as the target nucleic acid, or segment thereof. In certain embodiments the term “amplified” refers to a method that comprises a polymerase chain reaction (PCR). For example, an amplified product can contain one or more nucleotides more than the amplified nucleotide region of a nucleic acid template sequence (e.g., a primer can contain “extra” nucleotides such as a transcriptional initiation sequence, in addition to nucleotides complementary to a nucleic acid template gene molecule, resulting in an amplified product containing “extra” nucleotides or nucleotides not corresponding to the amplified nucleotide region of the nucleic acid template gene molecule). Accordingly, fragments can include fragments arising from segments or parts of amplified nucleic acid molecules containing, at least in part, nucleotide sequence information from or based on the representative nucleic acid template molecule.

Nucleic acid may be single or double stranded. Single stranded DNA, for example, can be generated by denaturing double stranded DNA by heating or by treatment with alkali, for example. In certain embodiments, nucleic acid is in a D-loop structure, formed by strand invasion of a duplex DNA molecule by an oligonucleotide or a DNA-like molecule such as peptide nucleic acid (PNA).

Determining Fetal Nucleic Acid Content. The amount of fetal nucleic acid (e.g., concentration, relative amount, absolute amount, copy number, and the like) in nucleic acid is determined in some embodiments. In certain embodiments, the amount of fetal nucleic acid in a sample is referred to as “fetal fraction”. In some embodiments “fetal fraction” refers to the fraction of fetal nucleic acid in circulating cell-free nucleic acid in a sample (e.g., a blood sample, a serum sample, a plasma sample) obtained from a pregnant female. In certain embodiments, the amount of fetal nucleic acid is determined according to markers specific to a male fetus (e.g., Y-chromosome markers; RhD marker in RhD-negative females), allelic ratios of polymorphic sequences, or according to one or more markers specific to fetal nucleic acid and not maternal nucleic acid (e.g., differential epigenetic biomarkers (e.g., methylation; described in further detail below) between mother and fetus, or fetal RNA markers in maternal blood plasma, see Lo, JOURNAL OF HISTOCHEMISTRY AND CYTOCHEMISTRY 2005, 53 (3): 293-296, incorporated by reference.

Determination of fetal nucleic acid content (e.g., fetal fraction) sometimes is performed using a fetal quantifier assay (FQA) as described, for example, in U.S. Patent Application Publication No. 2010/0105049, which is hereby incorporated by reference. This type of assay allows for the detection and quantification of fetal nucleic acid in a maternal sample based on the methylation status of the nucleic acid in the sample. In certain embodiments, the amount of fetal nucleic acid from a maternal sample can be determined relative to the total amount of nucleic acid present, thereby providing the percentage of fetal nucleic acid in the sample. In certain embodiments, the copy number of fetal nucleic acid can be determined in a maternal sample. In certain embodiments, the amount of fetal nucleic acid can be determined in a sequence-specific (or portion-specific) manner and sometimes with sufficient sensitivity to allow for accurate chromosomal dosage analysis (for example, to detect the presence or absence of a fetal aneuploidy).

A fetal quantifier assay (FQA) can be performed in conjunction with any of the methods described herein. Such an assay can be performed by any method known in the art and/or described in U.S. Patent Application Publication No. 2010/0105049 (incorporated by reference), such as, for example, by a method that can distinguish between maternal and fetal DNA based on differential methylation status, and quantify (e.g., determine the amount of) the fetal DNA. In certain embodiments, a fetal quantifier assay (FQA) can be used to determine the concentration of fetal DNA in a maternal sample, for example, by the following method: a) determine the total amount of DNA present in a maternal sample; b) selectively digest the maternal DNA in a maternal sample using one or more methylation sensitive restriction enzymes thereby enriching the fetal DNA; c) determine the amount of fetal DNA from step b); and d) compare the amount of fetal DNA from step c) to the total amount of DNA from step a), thereby determining the concentration of fetal DNA in the maternal sample. In certain embodiments, the absolute copy number of fetal nucleic acid in a maternal sample can be determined, for example, using mass spectrometry and/or a system that uses a competitive PCR approach for absolute copy number measurements. See for example, Ding and Cantor (2003) PNAS, USA 100:3059-3064, and U.S. Patent Application Publication No. 2004/0081993, both of which are hereby incorporated by reference.

In certain embodiments, fetal fraction can be determined based on allelic ratios of polymorphic sequences (e.g., single nucleotide polymorphisms (SNPs)), such as, for example, using a method described in U.S. Patent Application Publication No. 2011/0224087, which is hereby incorporated by reference. In such a method, nucleotide sequence reads are obtained for a maternal sample and fetal fraction is determined by comparing the total number of nucleotide sequence reads that map to a first allele and the total number of nucleotide sequence reads that map to a second allele at an informative polymorphic site (e.g., SNP) in a reference genome. In certain embodiments, fetal alleles are identified, for example, by their relative minor contribution to the mixture of fetal and maternal nucleic acids in the sample when compared to the major contribution to the mixture by the maternal nucleic acids. Accordingly, the relative abundance of fetal nucleic acid in a maternal sample can be determined as a parameter of the total number of unique sequence reads mapped to a target nucleic acid sequence on a reference genome for each of the two alleles of a polymorphic site.

The amount of fetal nucleic acid in extracellular nucleic acid can be quantified and used in conjunction with a method provided herein. Thus, in certain embodiments, methods of the technology described herein comprise an additional step of determining the amount of fetal nucleic acid. The amount of fetal nucleic acid can be determined in a nucleic acid sample from a subject before or after processing to prepare sample nucleic acid. In certain embodiments, the amount of fetal nucleic acid is determined in a sample after sample nucleic acid is processed and prepared, which amount is utilized for further assessment. In some embodiments, an outcome comprises factoring the fraction of fetal nucleic acid in the sample nucleic acid (e.g., adjusting counts, removing samples, making a call or not making a call). In certain embodiments, a method provided herein can be used in conjunction with a method for determining fetal fraction. For example, methods for determining fetal fraction that include a normalization process may comprise one or more normalization methods provided herein (e.g., a principal component normalization).

The determination step can be performed before, during, at any one point in a method described herein, or after certain (e.g., aneuploidy detection, fetal gender determination) methods described herein. For example, to achieve a fetal gender or aneuploidy determination method with a given sensitivity or specificity, a fetal nucleic acid quantification method may be implemented prior to, during or after fetal gender or aneuploidy determination to identify those samples with greater than about 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25% or more fetal nucleic acid. In some embodiments, samples determined as having a certain threshold amount of fetal nucleic acid (e.g., about 15% or more fetal nucleic acid; about 4% or more fetal nucleic acid) are further analyzed for fetal gender or aneuploidy determination, or the presence or absence of aneuploidy or genetic variation, for example. In certain embodiments, determinations of, for example, fetal gender or the presence or absence of aneuploidy are selected (e.g., selected and communicated to a patient) only for samples having a certain threshold amount of fetal nucleic acid (e.g., about 15% or more fetal nucleic acid; about 4% or more fetal nucleic acid).

In some embodiments, the determination of fetal fraction or determining the amount of fetal nucleic acid is not required or necessary.

Enriching Nucleic Acids. In some embodiments, nucleic acid (e.g., extracellular nucleic acid) is enriched or relatively enriched for a subpopulation or species of nucleic acid. Nucleic acid subpopulations can include, for example, fetal nucleic acid, maternal nucleic acid, nucleic acid comprising fragments of a particular length or range of lengths, or nucleic acid from a particular genome region (e.g., single chromosome, set of chromosomes, and/or certain chromosome regions). Such enriched samples can be used in conjunction with a method provided herein. Thus, in certain embodiments, methods of the technology comprise an additional step of enriching for a subpopulation of nucleic acid in a sample, such as, for example, fetal nucleic acid. In certain embodiments, a method for determining fetal fraction described above also can be used to enrich for fetal nucleic acid. In certain embodiments, maternal nucleic acid is selectively removed (partially, substantially, almost completely or completely) from the sample. In certain embodiments, enriching for a particular low copy number species nucleic acid (e.g., fetal nucleic acid) may improve quantitative sensitivity. Methods for enriching a sample for a particular species of nucleic acid are described, for example, in U.S. Pat. No. 6,927,028, International Patent Application Publication No. WO2007/140417, International Patent Application Publication No. WO2007/147063, International Patent Application Publication No. WO2009/032779, International Patent Application Publication No. WO2009/032781, International Patent Application Publication No. WO2010/033639, International Patent Application Publication No. WO2011/034631, International Patent Application Publication No. WO2006/056480, and International Patent Application Publication No. WO2011/143659, the entire content of each is incorporated herein by reference, including all text, tables, equations and drawings.

In some embodiments, nucleic acid is enriched for certain target fragment species and/or reference fragment species. In certain embodiments, nucleic acid is enriched for a specific nucleic acid fragment length or range of fragment lengths using one or more length-based separation methods described below. In certain embodiments, nucleic acid is enriched for fragments from a select genomic region (e.g., chromosome) using one or more sequence-based separation methods described herein and/or known in the art. Certain methods for enriching for a nucleic acid subpopulation (e.g., fetal nucleic acid) in a sample are described in detail below.

Some methods for enriching for a nucleic acid subpopulation (e.g., fetal nucleic acid) that can be used with a method described herein include methods that exploit epigenetic differences between maternal and fetal nucleic acid. For example, fetal nucleic acid can be differentiated and separated from maternal nucleic acid based on methylation differences. Methylation-based fetal nucleic acid enrichment methods are described in U.S. Patent Application Publication No. 2010/0105049, which is incorporated by reference herein. Such methods sometimes involve binding a sample nucleic acid to a methylation-specific binding agent (methyl-CpG binding protein (MBD), methylation specific antibodies, and the like) and separating bound nucleic acid from unbound nucleic acid based on differential methylation status. Such methods also can include the use of methylation-sensitive restriction enzymes (as described above; e.g., HhaI and HpaII), which allow for the enrichment of fetal nucleic acid regions in a maternal sample by selectively digesting nucleic acid from the maternal sample with an enzyme that selectively and completely or substantially digests the maternal nucleic acid to enrich the sample for at least one fetal nucleic acid region.

Another method for enriching for a nucleic acid subpopulation (e.g., fetal nucleic acid) that can be used with a method described herein is a restriction endonuclease enhanced polymorphic sequence approach, such as a method described in U.S. Patent Application Publication No. 2009/0317818, which is incorporated by reference herein. Such methods include cleavage of nucleic acid comprising a non-target allele with a restriction endonuclease that recognizes the nucleic acid comprising the non-target allele but not the target allele; and amplification of uncleaved nucleic acid but not cleaved nucleic acid, where the uncleaved, amplified nucleic acid represents enriched target nucleic acid (e.g., fetal nucleic acid) relative to non-target nucleic acid (e.g., maternal nucleic acid). In certain embodiments, nucleic acid may be selected such that it comprises an allele having a polymorphic site that is susceptible to selective digestion by a cleavage agent, for example.

Some methods for enriching for a nucleic acid subpopulation (e.g., fetal nucleic acid) that can be used with a method described herein include selective enzymatic degradation approaches. Such methods involve protecting target sequences from exonuclease digestion thereby facilitating the elimination in a sample of undesired sequences (e.g., maternal DNA). For example, in one approach, sample nucleic acid is denatured to generate single stranded nucleic acid, single stranded nucleic acid is contacted with at least one target-specific primer pair under suitable annealing conditions, annealed primers are extended by nucleotide polymerization generating double stranded target sequences, and digesting single stranded nucleic acid using a nuclease that digests single stranded (e.g., non-target) nucleic acid. In certain embodiments, the method can be repeated for at least one additional cycle. In certain embodiments, the same target-specific primer pair is used to prime each of the first and second cycles of extension, and In certain embodiments, different target-specific primer pairs are used for the first and second cycles.

In some embodiments, a selective nucleic acid capture process is used to separate target and/or reference fragments away from the nucleic acid sample. Commercially available nucleic acid capture systems include, for example, Nimblegen sequence capture system (Roche NimbleGen, Madison, Wis.); Illumina BEADARRAY® platform (Illumina, San Diego, Calif.); Affymetrix GENECHIP® platform (Affymetrix, Santa Clara, Calif); Agilent SureSelect Target Enrichment System (Agilent Technologies, Santa Clara, Calif.); and related platforms. Such methods typically involve hybridization of a capture oligonucleotide to a segment or all of the nucleotide sequence of a target or reference fragment and can include use of a solid phase (e.g., solid phase array) and/or a solution based platform. Capture oligonucleotides (sometimes referred to as “bait”) can be selected or designed such that they preferentially hybridize to nucleic acid fragments from selected genomic regions or loci (e.g., one of chromosomes 21, 18, 13, X or Y, or a reference chromosome). In certain embodiments, a hybridization-based method (e.g., using oligonucleotide arrays) can be used to enrich for nucleic acid sequences from certain chromosomes (e.g., from a Y or X chromosome) or segments of interest thereof.

In some embodiments, nucleic acid is enriched for a particular nucleic acid fragment length, range of lengths, or lengths under or over a particular threshold or cutoff using one or more length-based separation methods. Nucleic acid fragment length typically refers to the number of nucleotides in the fragment. Nucleic acid fragment length also is sometimes referred to as nucleic acid fragment size. In some embodiments, a length-based separation method is performed without measuring lengths of individual fragments. In some embodiments, a length based separation method is performed in conjunction with a method for determining length of individual fragments. In some embodiments, length-based separation refers to a size fractionation procedure where all or part of the fractionated pool can be isolated (e.g., retained) and/or analyzed. Size fractionation procedures are known in the art (e.g., separation on an array, separation by a molecular sieve, separation by gel electrophoresis, separation by column chromatography (e.g., size-exclusion columns), and microfluidics-based approaches). In certain embodiments, length-based separation approaches can include fragment circularization, chemical treatment (e.g., formaldehyde, polyethylene glycol (PEG)), mass spectrometry and/or size-specific nucleic acid amplification, for example.

Polymerase Chain Reaction (PCR). With polymerase chain reaction (PCR), it is possible to amplify a single copy of a specific target sequence in genomic DNA to a level that can be detected by several different methodologies (e.g., staining, hybridization with a labeled probe, incorporation of biotinylated primers followed by avidin-enzyme conjugate detection, incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

Amplification-based methods as disclosed herein typically include amplification of a multiple target nucleic acids by multiplex amplification (amplification of multiple target nucleic acids in parallel). In various embodiments, the nucleic acids are amplified, for example, from the sample or after isolation from the sample. Amplification refers to production of additional copies of a nucleic acid sequence and is generally conducted using polymerase chain reaction (PCR) or other technologies well-known in the art (e.g., Dieffenbach and Dveksler, PCR PRIMER, A LABORATORY MANUAL, 1995, Cold Spring Harbor Press, Plainview, N.Y.). The amplification reaction may be any amplification reaction known in the art that amplifies nucleic acid molecules, such as polymerase chain reaction, nested polymerase chain reaction, polymerase chain reaction-single strand conformation polymorphism, ligase chain reaction (Barany, F. GENOME RESEARCH, 1:5-16 (1991); Barany, F., PNAS, 88:189-193 (1991); U.S. Pat. Nos. 5,869,252; and 6,100,099), strand displacement amplification and restriction fragment length polymorphism, transcription based amplification system, rolling circle amplification, and hyper-branched rolling circle amplification. Further examples of amplification techniques that can be used include, without limitation, quantitative PCR, quantitative fluorescent PCR (QF-PCR), multiplex fluorescent PCR (MF-PCR), real time PCR (RTPCR), single cell PCR, restriction fragment length polymorphism (PCR-RFLP), RT-PCR-RFLP, hot start PCR, in situ polonony PCR, in situ rolling circle amplification (RCA), bridge PCR, picotiter PCR, and emulsion PCR. Other suitable amplification methods include transcription amplification, self-sustained sequence replication, selective amplification of target polynucleotide sequences, consensus sequence primed polymerase chain reaction (CP-PCR), arbitrarily primed polymerase chain reaction (AP-PCR), degenerate oligonucleotide-primed PCR (DOP-PCR) and nucleic acid based sequence amplification (NABSA). Other amplification methods that can be used herein include those described in U.S. Pat. Nos. 5,242,794; 5,494,810; 4,988,617; and 6,582,938.

In typical embodiments, the amplification reaction is the polymerase chain reaction. Polymerase chain reaction refers to methods by K. B. Mullis (U.S. Pat. Nos. 4,683,195 and 4,683,202, hereby incorporated by reference) for increasing concentration of segments of the four target sequences in a mixture of genomic DNA without cloning or purification.

Multiplex PCR is a molecular biology technique for amplification of multiple targets in a single PCR mix. In a multiplexing assay, more than one target sequence can be amplified by using multiple primer pairs in a reaction mixture. These multiplex PCR-based methods may be used to determine gender of a fetus in earlier terms of pregnancy. Male gender often renders a fetus more susceptible to X-linked disorders. The term multiplex-PCR refers to a single PCR reaction carried out on nucleic acid obtained from a single source (e.g., plasma or serum from a pregnant woman) using more than one primer set for the purpose of amplifying two or more DNA sequences in a single reaction. Multiplex polymerase chain reaction is another modification of polymerase chain reaction and is used in order to rapidly detect multiple gene sequences in a single PCR reaction. Multiplex PCR is typically accomplished using multiple primer sequences, each with a unique fluorophore for detection and quantification. This process amplifies DNA samples using the primers along with temperature-mediated DNA polymerases in a thermal cycler. Multiplex-PCR consists of multiple primer sets within a single PCR mixture to produce amplicons that are specific to different DNA sequences.

Target Y chromosome genes. Among the Y chromosome markers available, the inventors selected and investigated the use of target nucleotide sequences in the SRY, DAZ2 and TSPY and as an internal control, nucleotide sequences of the ACTB gene.

SRY. The SRY gene provides instructions for making a protein called the sex-determining region Y protein. The sex-determining region Y protein produced from this gene acts as a transcription factor, which means it attaches (binds) to specific regions of DNA and helps control the activity of particular genes. A reference polynucleotide sequence of the SRY gene is described by, and incorporated by reference to NCBI Reference Sequence: NG_011751.1. The SRY primers of the invention which were selected to bind to segments of the SRY gene are identified by SEQ ID NOS: 1 and 2 which produce an amplicon of 769 bp.

DAZ2 (deleted in azoospermia 2) is a protein coding gene. Diseases associated with DAZ2 include Spermatogenic Failure, Y-Linked, 2 and Azoospermia. Gene Ontology (GO) annotations related to this gene include nucleic acid binding and nucleotide binding. This gene is a member of the DAZ gene family and is a candidate for the human Y-chromosomal azoospermia factor (AZF). Its expression is restricted to premeiotic germ cells, particularly in spermatogonia. It encodes an RNA-binding protein that is important for spermatogenesis. Four copies of this gene are found on chromosome Y within palindromic duplications; one pair of genes is part of the P2 palindrome and the second pair is part of the P1 palindrome. Each gene contains a 2.4 kb repeat including a 72-bp exon, called the DAZ repeat; the number of DAZ repeats is variable and there are several variations in the sequence of the DAZ repeat. Each copy of the gene also contains a 10.8 kb region that may be amplified; this region includes five exons that encode an RNA recognition motif (RRM) domain. This gene contains one copy of the 10.8 kb repeat. Alternative splicing results in multiple transcript variants encoding different isoforms.

A reference polynucleotide sequence of the DAZ2 gene is described by, and incorporated by reference to NCBI Reference Sequence: NG_028267.1. The DAZ2 primers of the invention which were selected to bind to segments of the DAZ2 gene are identified by SEQ ID NOS: 7 and 8 which produce an amplicon of 269 bp.

DAZ2 is a member of the DAZ protein family a group of three highly conserved RNA-binding proteins that are important in gametogenesis and meiosis. Therefore, mutations in the genes that encode for the DAZ proteins can have detrimental consequences for fertility. The three members of the DAZ protein family include BOULE (BOLL), DAZL (DAZLA) and DAZ (DAZ1, DAZ2, DAZ3 and DAZ4). DAZ1 is located on the Y chromosome in higher primates and is important for spermatogenesis. BOULE and DAZL are important for both oogenesis and spermatogenesis. BOULE and DAZL are both located on autosomes as single copies. However DAZ is located with multiple copies in the Y chromosome only.

TSPY (DYS14). Testis-specific Y-encoded protein 1 is a protein that in humans is encoded by the TSPY1 gene. The protein encoded by this gene is found only in testicular tissue and may be involved in spermatogenesis. A reference polynucleotide sequences of the TSPY1 gene are described by, and incorporated by reference to NCBI Reference Sequence: NG_027958.1 DYS14 is located within the TSPY-encoding, gene. The TSPY1/DYS14 primers of the invention which were selected to bind to segments of the TSPY1 gene are identified by SEQ ID NOS: 5 and 6 which produce an amplicon of 329-356 bp.

ACTB. The ACTB gene provides instructions for making a protein called beta (β)-actin, which is part of the actin protein family. Proteins in this family are organized into a network of fibers called the actin cytoskeleton, which makes up the structural framework inside cells. A reference polynucleotide sequence of the ACTB gene is described by, and incorporated by reference to NCBI Reference Sequence: NG_007992.1. The ACTB primers of the invention which were selected to bind to segments of the ACTB gene are identified by SEQ ID NOS: 3 and 4 which produce an amplicon of 541 bp.

Primers. Primers, such as those designed by the inventors and disclosed herein, may be produced by a variety of methods including but not limited to direct chemical synthesis or cloning of appropriate sequences using methods well known in the art (Narang et al., METHODS ENZYMOL., 68:90 (1979); Brown et al., METHODS ENZYMOL., 68:109 (1979)). Primers can be custom made by commercial sources such as Operon Technologies, Amersham Pharmacia Biotech, Sigma, and Life Technologies. The primers can have an identical melting temperature. The lengths of the primers can be extended or shortened at the 5′ end or the 3′ end to produce primers with desired melting temperatures. Also, the annealing position of each primer pair can be designed such that the sequence and length of the primer pairs yield the desired melting temperature. The simplest equation for determining the melting temperature of primers smaller than 25 base pairs is the Wallace Rule (Td=2(A+T)+4(G+C)). Computer programs can also be used to help design primers, including but not limited to Array Designer Software from Arrayit Corporation (Sunnyvale, Calif), Oligonucleotide Probe Sequence Design Software for Genetic Analysis from Olympus Optical Co., Ltd. (Tokyo, Japan), NetPrimer, and DNAsis Max v3.0 from Hitachi Solutions America, Ltd. (South San Francisco, Calif). The TM (melting or annealing temperature) of each primer can be calculated using software programs such as OligoAnalyzer 3.1, available on the web site of Integrated DNA Technologies, Inc. (Coralville, Iowa).

Primer modifications. To decrease degradation or inactivation of primers or other oligonucleotides by nucleases, various modifications to the native phosphodiester oligodeoxyribonucleotides primers disclosed herein may be made. These include use of phosphorothioate (PS) bonds which substitute a sulfur atom for a non-bridging oxygen in the phosphate backbone of an oligonucleotide. Approximately 50% of the time (due to the 2 resulting stereoisomers that can form), PS modification renders the internucleotide linkage more resistant to nuclease degradation. Another modification involves use of 2′-O-Methyl (2′OMe). A naturally occurring post-transcriptional modification of RNA, 2′OMe is found in tRNA and other small RNAs. Oligonucleotides can be directly synthesized to contain 2′OMe. DNA oligonucleotides that include this modification are typically 5- to 10-fold less susceptible to DNases than unmodified DNA. The 2′OMe modification is commonly used in antisense oligonucleotides as a means to increase stability and binding affinity to target transcripts. Another modification involves use of 2′ fluoro bases which have a fluorine-modified ribose which increases binding affinity (T m) and also confers some relative nuclease resistance compared to native RNA. Preferably, this modification is used in conjunction with PS-modified bonds. Another modification involves incorporation of inverted dT and ddT. Inverted dT can be incorporated at the 3′ end of an oligonucleotide, leading to a 3′-3′ linkage that will inhibit degradation by 3′ exonucleases and extension by DNA polymerases. In addition, placing an inverted, 2′,3′ dideoxy-dT base (5′ Inverted ddT) at the 5′ end of an oligonucleotide prevents spurious ligations and may protect against some forms of enzymatic degradation. Phosphorylation of the 3′ end of oligonucleotides will inhibit degradation by some 3′-exonucleases. Use of a C3 Spacer may also be used. The phosphoramidite C3 Spacer can be incorporated internally, or at either end of an oligo. In some variant embodiments which use fluorophores for amplicon detection, a long hydrophilic spacer arm is introduced for the attachment of fluorophores or other pendent groups. The C3 spacer also can be used to inhibit degradation by 3′ exonucleases. This modification is just a short 3 carbon chain (C3), which is attached to the terminal 3′ hydroxyl group of the oligonucleotide.

In some embodiments, primers that vary from those of SEQ ID NOS: 1-6, for example, by 1, 2, 3, 4 or more deletions, substitutions or insertions of nucleotides, but which amplify detectable segments of the target genes comprising SEQ ID NOS: 7-10 or corresponding variants of SEQ ID NOS: 7-10 may be used for multiplex PCR identification of Y chromosome sequences. Such variant primers may produce the amplicons of SEQ ID NOS: 7-10 disclosed herein or amplicons comprising sequences at least 95, 96, 97, 98, 99, 99.5, 99.9% identical to SEQ ID NOS: 7-10. The length of such a variant PCR primer is typically long enough to specifically identify the target gene and short enough for the primer to easily bind to a polynucleotide template, for example, 15, 16, 17, 18, 19, 20, 21, 22, 23, 24, 25, 26, 27, 28, 29 or 30 nucleotides in length. In some embodiments a primer will have a melting temperature of 50, 51, 52, 53, 54, 55, 56, 57, 58, 59 or 60° C. In some embodiments a primer will have a GC content (%) of 30, 40, 50, 60, 70 or 80%. Preferably the Tₘ of two primers used for PCR will not differ by more than 1, 1.5 or 2° C. Preferably primers will not have sequences that form primer dimers or that form hairpin loops.

In some embodiments a primer may have a length of 15, 16, 17, 18, 19, 20, 21, 22, 23, 24, 25, 26, 27, 28, 29 or 30 nucleotides and/or have a GC content (%) of 30, 40, 50, 60, 70 or 80%; and/or have a melting temperature of about 5, 6, 7, 8, 9, or 10° C. above than of PCR primers or within a range of 68, 69 or 70° C. Probes and primers having the lengths and other features described above may be designed to amplify one or more portions of a target segment of a TAA polynucleotide or other segments of a TAA polynucleotide suitable for production and identification of an amplicon.

Amplicon length. Amplicon length produced by use of such primers may be selected based on efficiency of amplification of a target polynucleotide sequence. Usually a short amplicon length is preferred. In some embodiments amplicon length ranges from 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, or 300 nucleotides. A preferred range of amplicon lengths is disclosed in the Figures and include those lengths produced using the primers of SEQ ID NOS: 1-8.

Measuring amplicon length. Agarose gel electrophoresis is the easiest and most common way of separating and analyzing DNA. Agarose gels are prepared to inspect the DNA, to quantify it, or to isolate or size a particular band. The DNA can be visualized in the gel by addition of ethidium bromide, which is mutagenic, or less-toxic proprietary dyes such as GelRed, GelGreen, and SYBR Safe. Ethidium bromide and the proprietary dyes bind to DNA and are fluorescent, meaning that they absorb invisible UV light and transmit the energy as visible light. Most agarose gels are made between 0.7% and 2%. A 0.7% gel will show good separation (resolution) of large DNA fragments (5-10 kb) and a 2% gel will show good resolution for small fragments (0.2-1 kb). Some gels as high as 3% agarose are used for separating very tiny fragments but a vertical polyacrylamide gel is more appropriate in this case. Low percentage gels are very weak and may break when you try to lift them. High percentage gels are often brittle and do not set evenly.

While agarose gels can be used to resolve large fragments of DNA, polyacrylamide gels are used to separate shorter nucleic acids, generally in the range of 1-1000 base pairs, based on the concentration used. These gels can be run with or without a denaturant. Gels that are run without a denaturant are referred to as native gels. The DNA or RNA will migrate at different rates, depending on its secondary structure. Native gels allow the DNA or RNA to remain double stranded. Adding a denaturant to the gel, such as urea, will generally make all of the nucleic acids single stranded. Secondary structure will not form in denaturing gels and, therefore, only the length of the DNA will affect mobility.

In some alternative embodiments, amplicons from the four target genes may be detected using labeled probes having different colors or different detectable moieties that are sufficiently complementary and hybridize to the amplified products corresponding to the target nucleic acid. Thus, the presence, amount, and/or identity of the amplified product can be detected by hybridizing a labeled probe, such as a fluorescently-labeled probe, that is complementary to the amplified product.

First, second and third trimesters. The first trimester is the earliest phase of pregnancy. It starts on the first day of the mother's last period—before you're even actually pregnant—and lasts until the end of the 13th week. It's a time of great anticipation and of rapid changes for the mother and baby. The methods disclosed herein can detect fetal gender prior to the end of the first trimester, for example, by the <8, 8, 9, 10, 11, 12, or 13^{th} week. It may also detect fetal gender after the 13, 14, 15, 16, 17, 18 or >18^{th} week of pregnancy. The second trimester begins at about 14 weeks and the third trimester at about 28 weeks. The methods disclosed herein are useful for early detection of fetal gender, but may also be used in the second and third trimesters of pregnancy. In some alternative embodiments, the methods disclosed herein may be used to determine gender of an individual at any time of life or for forensic investigation or characterization of the gender of a living or dead subject.

X-linked disorders, the diagnosis of which benefits from determination of gender, include Adrenoleukodystrophy, Aldred syndrome, Becker muscular dystrophy, Color blindness, Creatine transporter defect, Duchenne muscular dystrophy, Endocardial fibroelastosis, Fabry disease, FG syndrome, Haemophilia, Haemophilia A, Haemophilia B, Hoyeraal-Hreidarsson syndrome, Spinal and bulbar muscular atrophy, L1 syndrome, Lysosomal storage disease, MASA syndrome, McLeod syndrome, Menkes disease, Nasodigitoacoustic syndrome, Norrie disease, Occipital horn syndrome, Ocular albinism, Ocular albinism type 1, Oculocerebrorenal syndrome, Ornithine transcarbamylase deficiency, Oto-palato-digital syndrome, Papillary fibroelastoma, Pelizaeus-Merzbacher disease, Renpenning's syndrome, Say-Meyer syndrome, Simpson-Golabi-Behmel syndrome, Smith-Fineman-Myers syndrome, Wieacker syndrome, X-linked agammaglobulinemia, Template:X-linked disorders, X-linked dystonia parkinsonism, X-linked mental retardation, X-linked recessive chondrodysplasia punctata, X-linked spinal muscular atrophy type 2, or XMEN disease. Upon identification of gender of a fetus, parents and medical workers can determine a suitable course of action which for some X-linked disorders may involve treatment to reverse their symptoms or course of disease.

In some embodiments, the methods as disclosed herein may be used to detect gender of a subject having a disorder affecting sexual differentiation. Disorders of sexual development (DSD) encompass a group of congenital conditions associated with atypical development of internal and external genital structures. These conditions can be associated with variations in genes, developmental programming, and hormones. Affected individuals may be recognized at birth due to ambiguity of the external genitalia. Others may present later with postnatal virilization, delayed/absent puberty, or infertility. In some embodiments, maternal carriers of congenital adrenal hyperplasia when the fetus is female, may be treated with a steroid as early as possible to prevent virilization of the fetus. In other embodiments, gender determination in pregnancies with a high risk of X-linked diseases help decision makers to decide whether to keep the fetus or in some cases terminal a fetus with a male gender.

Treatments. Based on the identification of gender and/or the genetic profiles of each parent, the results of the methods disclosed herein may be used to select a treatment. In some countries, whether a pregnancy is continued is determined, at least in part, by determination of the fetal gender. Gender may also be used to select a prenatal regimen or treatment for a pregnant woman or a fetus based on gender, for example, providing a maternal diet with supplemental amino acids or energy sources (e.g., glucose, sugars) for a male fetus. Other prenatal treatments include symptomatic treatment, such a treatment of prenatal anemia with a fetal blood transfusion; supplementation therapy, such as administration of thyroxin or other enzymes or hormones; or in utero stem cell transplantation to correct metabolic or immunological defects.

Kits. As used herein, the term “kit” refers to any delivery system for delivering materials. In the context of PCR-based assays, such delivery systems include systems that allow for the storage, transport, or delivery of reaction reagents (e.g., plasma, serum, tissue, oligonucleotides, enzymes, etc. in the appropriate containers) and/or supporting materials (e.g., buffers, written instructions for performing the assay etc.) from one location to another. For example, kits include one or more enclosures (e.g., boxes or packages) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in the PCR, while a second container contains oligonucleotides, such as the primer pairs disclosed herein. The term “fragmented kit” is intended to encompass kits containing Analyte specific reagents (ASR's) regulated under section 520(e) of the Federal Food, Drug, and Cosmetic Act, but are not limited thereto. Any delivery system comprising two or more separate containers that each contains a subportion of the total kit components are included in the term “fragmented kit.” In contrast, a “combined kit” refers to a delivery system containing all of the components of a reaction assay in a single container (e.g., in a single box or package housing each of the desired components). The term “kit” includes both fragmented and combined kits.

Embodiments of this technology include, but are not limited to, the following.

A multiplex method for simultaneous detection of Y chromosome-specific target DNA of the SRY, DAZ2 and TSPY1 genes and control DNA of the ACTB gene in a sample containing DNA, comprising:
(a) simultaneously amplifying portions of the SRY, DAZ2, TSPY1 and ACTB to produce up to four different amplicons; wherein each amplicon has a different distinguishable length;
(b) detecting the presence or absence of amplicons from the SRY, DAZ2 and TSPY1 and ACTB by determining the length of each amplicon, selecting a sample containing Y chromosome DNA when presence of amplicons for at least two of SRY, DAZ2 or TSPY1 is detected, or selecting a sample containing Y chromosome DNA when presence of one or more amplicons from SRY, DAZ2 or TSPY1 having an intensity of ≥1,000,000 intensity is detected; or selecting a sample not containing Y chromosome DNA when no amplicons from at least two of SRY, DAZ2 or TSPY1 are detected or when only a presence of a single amplicon from SRY, DAZ2 or TSPY1 having an intensity lower than 1,000,000 is detected; and, optionally, sequencing the amplicons to confirm their origination from segments of SRY, DAZ2, TSPY1 and/or ACTB.

In some embodiments, the disclosed method uses a sample that was previously frozen, desiccated, dried, stored, or degraded DNA. In other embodiments, a fresh sample or a sample obtained less than 1, 2, 3, 4, 5, 6, or 7 days prior to use in PCR is used.

In preferred embodiments, the sample comprises fetal DNA. Fetal DNA may be present in maternal plasma, urine or other biological fluids, in amniotic fluid or in fetal tissues. Fetal DNA may be cell-free or present in fetal cells, including immature red blood cells that express CD71 or other fetal cell markers. Other fetal cell the protein products of the MMP14, MCAM, KCNQ4, CLDN6, and F3 genes and the corresponding genes encoding them. Fetal cell markers and antibodies to them may be used for fetal cell enrichment, for example, by affinity purification or cell sorting of fetal cells expressing these markers.

In some embodiments, the sample comprises maternal cell-free plasma or maternal cell-free serum. In other embodiments the sample comprises peripheral blood monocytes that are CD71 positive. In other embodiments, the sample comprises maternal urine. In some embodiments, the cord blood, amniotic fluid, fetal tissue or fetal blood any of which may be further purified to extract fetal DNA.

In some embodiments of the method, maternal or paternal DNA is used as the sample for PCR, typically in parallel or conjunction with detection of SRY, DAZ2, TSPY1 and ACTB in fetal DNA. In such embodiments, the method disclosed above for detecting markers in fetal DNA further comprises isolating DNA from the mother's buccal cells or from the father's blood or buccal cells or other parental biological fluids or tissues; and (a) simultaneously amplifying portions of the SRY, DAZ2, TSPY1 and ACTB to produce amplicons; wherein each amplicon has a different distinguishable length; and (b) detecting the presence or absence of amplicons from the SRY, DAZ2 and TSPY1 and ACTB by determining the length of each amplicon.

Some preferred embodiments, further comprise isolating the target DNA from the cellular, protein, and other components of the biological sample. Such embodiments, may further comprise contacting a sample with sequence-specific DNA probes to capture and subsequently elute away from non-captured DNA segments of SRY, DAZ2 or TSPY1 or ACTB for amplification. Reagents for capture include nucleic acid sequences complementary to the SRY, DAZ2 or TSPY1 or ACTB DNA targets and/or nucleic acid sequences complementary to the nucleic acid sequences of the primers disclosed herein.

In a preferred embodiment, the four sets of primers used for multiplex PCR comprise a first primer comprising SEQ ID NO:1 and a second primer comprising SEQ ID NO:2 which amplify the SRY target nucleic acid sequence; a third primer comprising SEQ ID NO: 7 and a fourth primer comprising SEQ ID NO:8 which amplify the DAZ2 target nucleic acid sequence; a fifth primer comprising SEQ ID NO:5 and a sixth primer comprising SEQ ID NO:6 which amplify the TSPY1 target nucleic acid sequence; and a seventh primer comprising SEQ ID NO:3 and an eighth primer consisting of SEQ ID NO: 4 which amplify the ACTB target nucleic acid sequence.

In one preferred embodiment of this multiplex method, the four sets of primers comprise, consist essentially of, or consist of a first primer comprising SEQ ID NO:1 and a second primer comprising SEQ ID NO:2 which amplify the SRY target nucleic acid sequence; a third primer comprising SEQ ID NO: 7 and a fourth primer comprising SEQ ID NO:8 which amplify the DAZ2 target nucleic acid sequence; a fifth primer comprising SEQ ID NO:5 and a sixth primer comprising SEQ ID NO:6 which amplify the TSPY1 target nucleic acid sequence; and a seventh primer comprising SEQ ID NO:3 and an eighth primer consisting of SEQ ID NO: 4 which amplify the ACTB target nucleic acid sequence; wherein one, two, three of four of said primers comprise one or more modified nucleotides selected from the group consisting of 2′-O-methyl nucleotides, 2′-fluoro-nucleotides, 2′-amino nucleotides, and arabinose nucleotides. In alterative embodiments, other modifications to the primer DNA may be made which do not substantially affect their capacity to amplify target DNAs, but which improve their stability, sensitivity, and/or prevent degradation.

In one embodiment of the methods disclosed herein one or both parents of a fetus from which a sample is obtained have or are carriers of an X-linked genetic disease, disorder or condition.

In another embodiment of the methods disclosed herein, the karyotype of the fetus is determined by methods known in the art. To make a karyotype, one takes a picture of the chromosome from one cell, cut out the images of the chromosomes, and arranges them using size, banding pattern, and centromere position as guides.

The multiplex methods disclosed herein may further comprise testing the fetus for muscular dystrophy, fragile X syndrome, or hemophilia; for X-linked lymphoproliferative syndrome, or for other genetic, preferably X-linked diseases, disorders or conditions.

In preferred embodiments, a male fetus is treated. In other embodiments, a female fetus is treated.

Another embodiment of this technology is directed to kit for determining gender of a biological sample comprising a set of primers that amplify segments of SRY, DAZ2, TSPY1 and ACTB genes to produce amplicons of different lengths; and, optionally one or more reagents required for PCR, reaction containers, packaging materials, and instructions. In one preferred embodiment, the kit comprises a first primer comprising SEQ ID NO:1 and a second primer comprising SEQ ID NO:2 which amplify the SRY target nucleic acid sequence; a third primer comprising SEQ ID NO: 7 and a fourth primer comprising SEQ ID NO:8 which amplify the DAZ2 target nucleic acid sequence; a fifth primer comprising SEQ ID NO:5 and a sixth primer comprising SEQ ID NO:6 which amplify the TSPY1 target nucleic acid sequence; and a seventh primer comprising SEQ ID NO:3 and an eighth primer consisting of SEQ ID NO: 4 which amplify the ACTB target nucleic acid sequence; and optionally, wherein one or more of said primers comprise one or more modified nucleotides selected from the group consisting of 2′-O-methyl nucleotides, 2′-fluoro-nucleotides, 2′-amino nucleotides, and arabinose nucleotides.

### Examples

The assay developed here is a sensitive and specific multiplex PCR that can detect male fetal DNA from any source of samples.

Primer design. Primer BLAST was used to identify putative primers. These primers were subsequently tested for specificity and sensitivity for detecting Y chromosome DNA and were synthesized by Integrated DNA Technologies, Inc. (Coralville, Iowa, USA).

All eight primers were manually strengthened by adjusting the annealing location and melting temperature to avoid nonspecific annealing to genes other than the specific gene.

TABLE 1
Primers designed for the amplification of the male specific SRY, DAZ2 and
TSPY1 genes and ACTB gene.

| S. | | Primer | | Amplicon |
|---|---|---|---|---|
| No | Gene | name | Sequence (5′-3′) | (bp) |
| 1 | SRY | SRYaF | TGTTGAGGGCGGAGAAATGCAAGTTTCATTACAA | 769 |
| 2 | | SRYaR | ATGTTACCCGATTGTCCTACAGCTTTGTCCAGTGG | |
| 3 | ACTB | ActinF | ACTTTCTGCATGTCCCCCGTCTGGC | 541 |
| 4 | | ActinR | GCCGGGAGACAGTCTCCACTCACC | |
| 5 | TSPY1 | DYS14F | GGTGCCAGAGAGGCTGCGGCA | 329-356 |
| 6 | | DYS14R | TAACCGGCTCCAGCTCCACCTGAA | |
| 7 | DAZ2 | DAZ2aF | GAAAATTTGTAGAACAGAGACAGAAATGCTTTGCTGTTA | 269 |
| 8 | | DAZ2aR | CCCGAACCAGAATATATCCAGAAGTCAGCAATTTAT | |

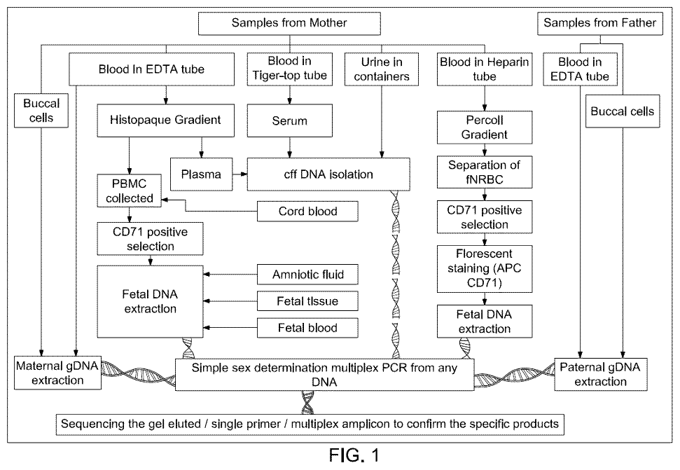

Collection of samples. All samples were collected at King Fahad Hospital of the University, Khobar, Saudi Arabia. Various sources of cffDNA and fetal DNA are presented in FIG. 1.

Maternal blood and cord blood samples were collected in lithium heparin vacutainer from women at delivery.

Samples were processed within 8-10 hours of collection for cffDNA extraction.

Serum samples were collected in tubes without clot activator or gel, and urine samples collected in sterile containers.

Samples then were centrifuged at 3000 rpm for 10 min in 20-24° C. twice to remove cells, then stored at −20° C. for cffDNA extraction.

Tissue samples, which were separated from a miscarriage at the 9^{th} week of pregnancy, cautiously from maternal tissue to avoid contamination. Blood samples were collected from the fetus. Miscarriage sample was collected in RNAPROTECT® cell reagent (Qiagen, Hilden, Germany).

Fetal Cell Isolation. Maternal cord blood samples were subjected to density gradient centrifugation using HISTOPAQUE®-1077.

Centrifugation was performed at 1430 rpm for 45 min.

The plasma was collected in 1.5 ml tubes and cell layer between plasma and HISTOPAQUE® was recovered then washed with PBS.

For positive selection of anti-CD71, cells were incubated for 30 min at 4-8° C. with a 1:10 CD71 MicroBeads, human (Miltenyi Biotec, Bergisch Gladbach, Germany).

Magnetic selection was achieved using the mini-MACS system (Miltenyi Biotec) according to the manufacturer's instructions.

DNA Extraction. DNA from fetal cells was extracted using the QIAamp DNA Blood Mini Kit® (Qiagen) according to manufacturer instructions.

DNA from fetal tissue was extracted using Puregene Cell and Tissue Kit® (Qiagen) according to manufacturer instructions.

cffDNA from maternal samples plasma, serum and urine were extracted using the QIAAMP MINELUTE CCFDNA MINI KIT® (Qiagen) according to manufacturer instructions.

Gender specific Multiplex PCR. Individual PCs for the amplicons as listed in the Table 1 were completed before designing the multiplex recipe. The PCR amplification reactions were set a total volume of 25 μl containing:
Absolute master mix 12.5 μl (MOLEQULE-ON®, Auckland, New Zealand),
Primer 1 μl (10 nM) SRYaF
Primer 1 μl (10 nM) SRYaR
Primer 1 μl (10 nM) ActinF
Primer 1 μl (10 nM) ActinR
Primer 1 μl (10 nM) DYS14F
Primer 1 μl (10 nM) DYS14R
Primer 1 μl (10 nM) DAZ2aF
Primer 1 μl (10 nM) DAZ2aR
Template DNA 25 ng
Distilled H₂O to 25 μl.

The PCR thermal cycling for the multiplex was as follows:
Step 1: 95° C. for 10 mins.
Step 2: 95° C. for 1 minute.
Step 3: 70° C. for 1 minute.
Step 4: 72° C. for 1 minute.
Step 5: Go to step 2, 35 cycles
Step 6: 72° C. for 5 minutes.
Step 7: Store at 4° C.

Amplicons were visualized using 2% agarose electrophoresis run at 100 volt for one hour.

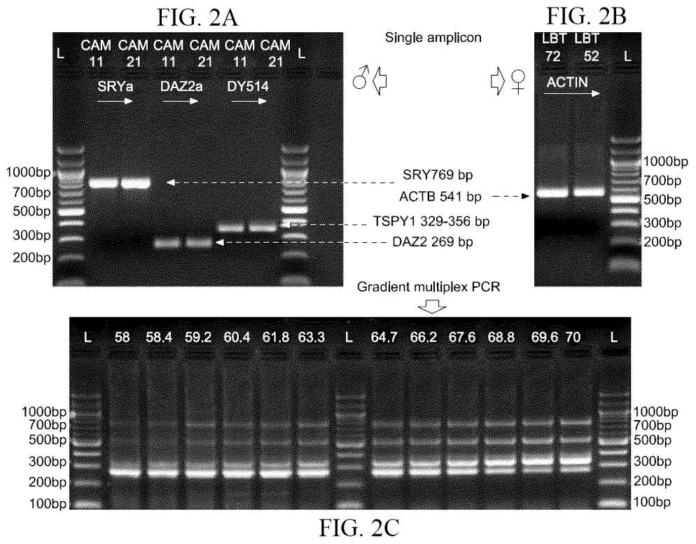

The designed primer pairs for SRY, ACTB, DAZ2 and TSPY1 genes (Table 1) were tested for the specific identification of the respective genes (FIGS. 2A-2B).

All four pairs (Table 1) of the primers were used to amplify target DNAs individually to confirm that the resulting amplicons were free from non-specific amplicons (FIGS. 2A and 2B).

The temperature gradient multiplex PCR was done to confirm the annealing temperature (59° C.) with specific amplicons (FIG. 2C).

Amplicons of concentration dependent multiplex PCR confirmed the stable specific products in various concentrations.

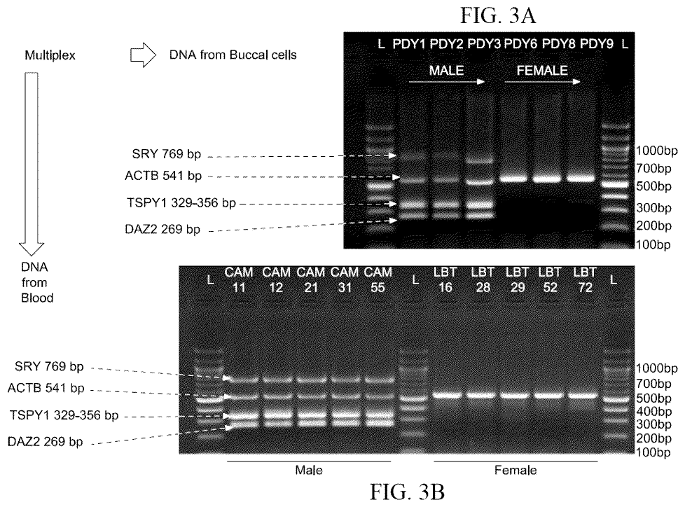

Multiplex PCR-based amplification from various samples like paternal DNA from father's blood, maternal DNA from mother's blood, paternal DNA from father's buccal cells, maternal DNA from mother's buccal cells, cffDNA from maternal urine, cffDNA from maternal serum, cffDNA from maternal plasma, cffDNA from cord blood, fetal DNA from fetal blood and fetal DNA from fetal tissue, confirmed the specificity (FIGS. 3A-3B).

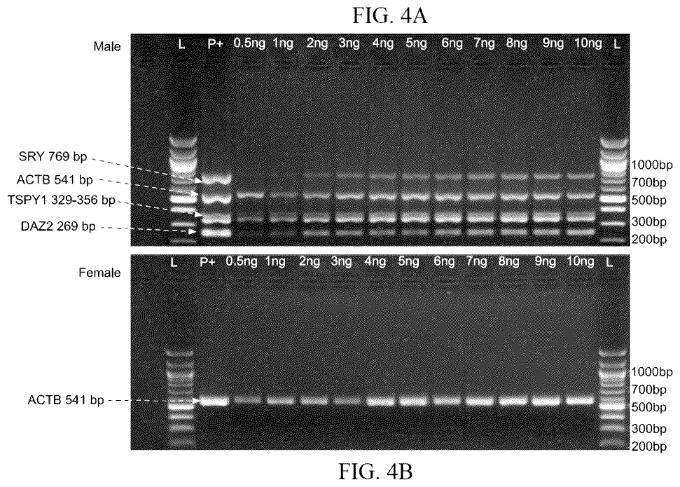

FIG. 4A shows multiplex PCR results after amplification of SRY, ACTB, TSPY1 and DAZ2 using different concentrations of male target DNA. Bands from Y chromosome markers are detectable at the lowest amount tested 0.5 ng. FIG. 4B shows multiplex PCR results after amplification of ACTB using different concentrations of female target DNA. Bands from ACTB are detectable at the lowest concentration tested 0.5 ng.

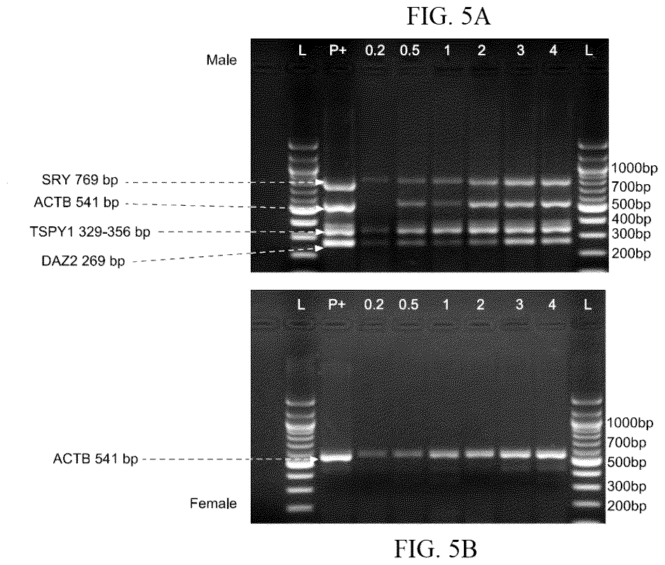

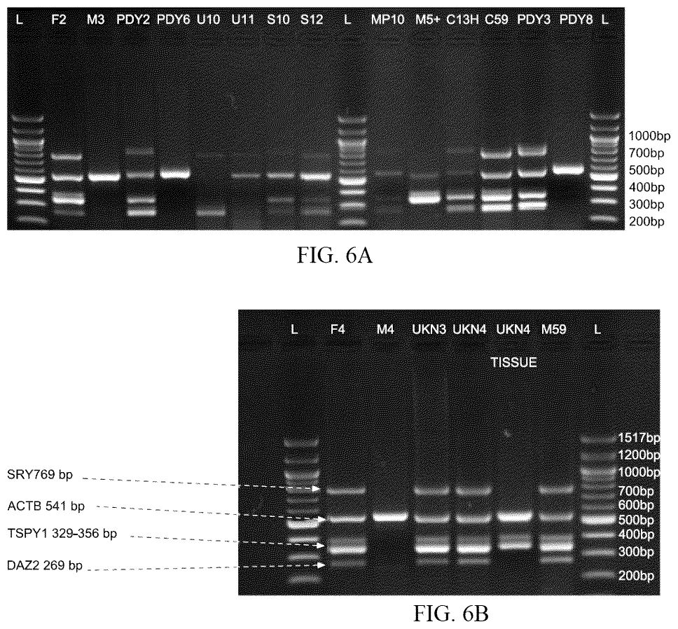

FIG. 5A shows multiplex PCR results after amplification of SRY, ACTB, TSPY1 and DAZ2 using different amounts of male target DNA. Bands from Y chromosome markers are detectable at the lowest amount tested 0.2 ng. FIG. 5B shows multiplex PCR results after amplification of ACTB using different amounts of female target DNA. Bands from ACTB are detectable at the lowest amount tested 0.2 ng. The sensitivity of the methods as disclosed herein distinguishes it from other PCR methods which require higher amounts of DNA. Some of the amplicons were very faint and their presence could not be conclusively determined. However, presence of two amplicons out of three Y markers was considered positive for identification of male gender.

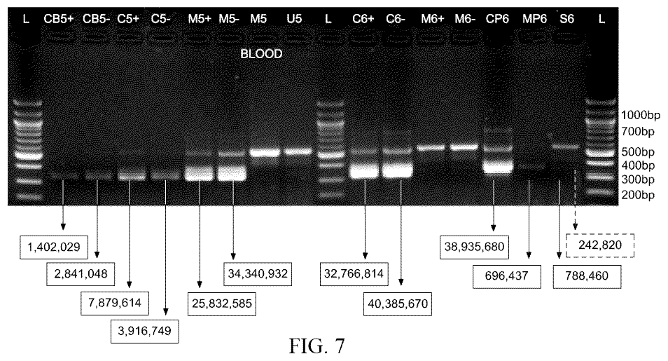

In order to confirm the presence of amplicons and to avoid human intervention when deciding the presence or absence of an amplicon, the intensity of amplicons of single Y marker were calculated using labimage (FIG. 7). Single Y marker amplicon with ≥1,000,000 intensity was considered to be positive for male gender.

Intensity may be determined or calculated by methods known in the art such as by using labimage software; see Alvarez-Venegas, et al., PNAS Apr. 11, 2006 103 (15) 6049-6054 (incorporated by reference).

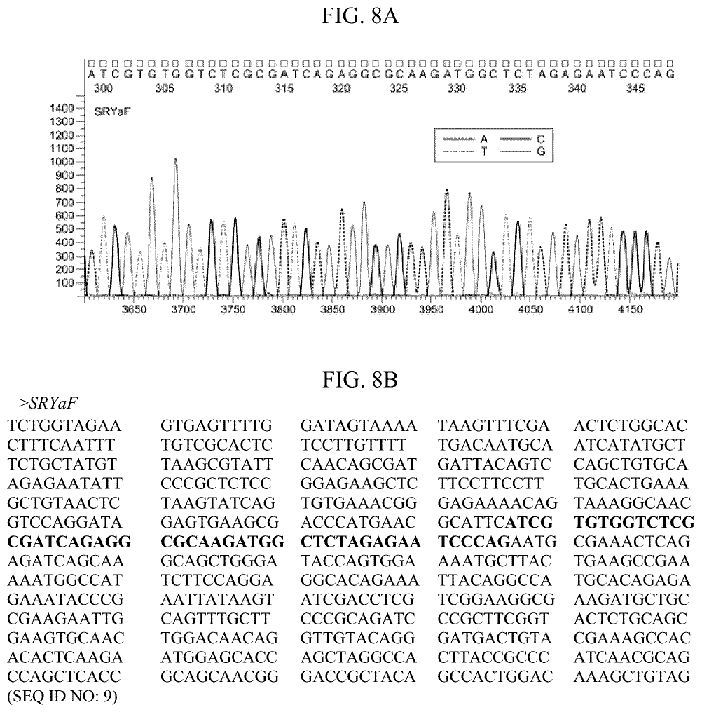

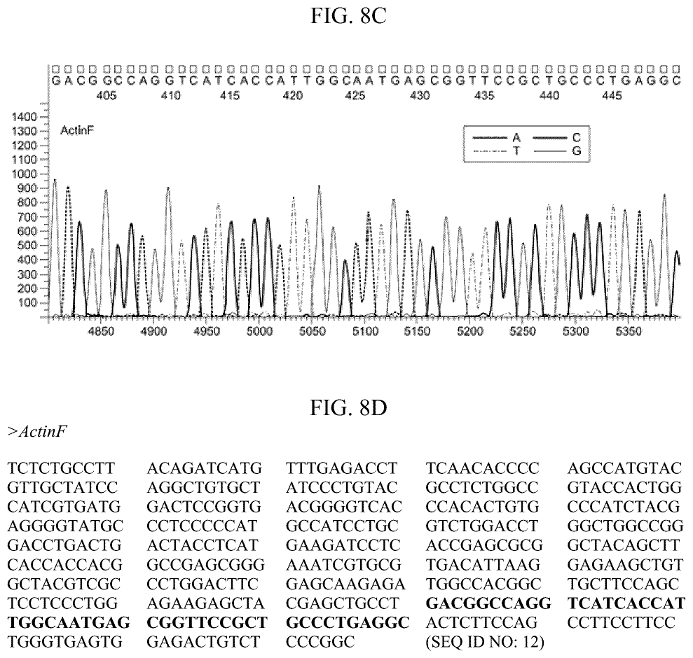

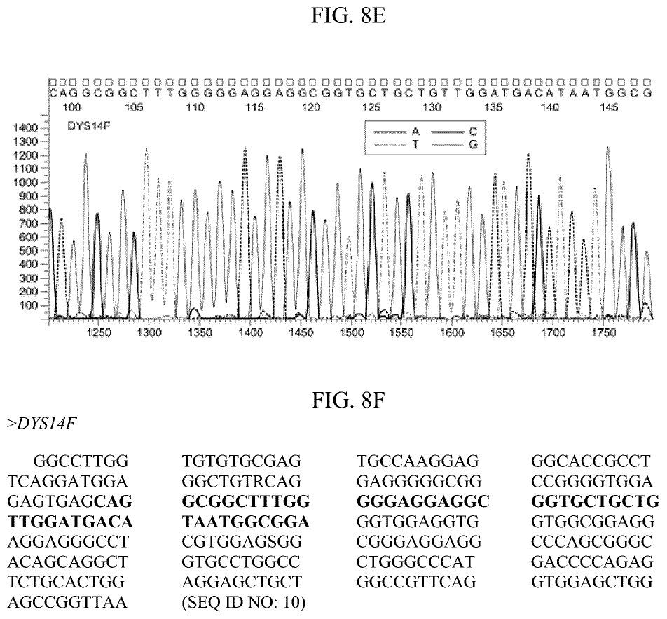

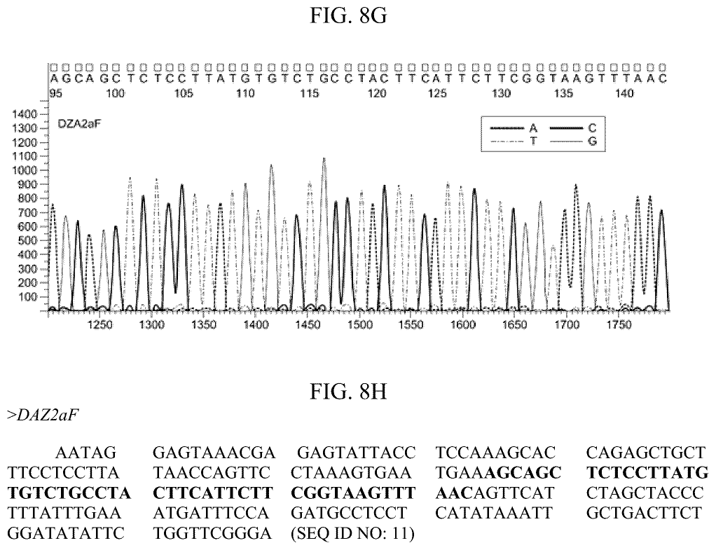

Using direct sequencing, individual PCR amplicons and gel-eluted amplicons from multiplex PCR were confirmed for the specific products (FIGS. 8A-8H). The gel-eluted amplicons of SRY, ACTB, DAZ2 and TSPY1 genes from multiplex PCR clearly indicated the absence of background noise.

The multiplex PCR as disclosed herein detects three male-specific genes: SRY, DAZ2 and TSPY1, which are biomarkers for the Y chromosome. As an internal control, it detects the ACTB gene from chromosome 7.

The eight pairs of primers designed and confirmed herein are highly sensitive, unique, and well matched for the simultaneous multiplex detection of the male-specific Y biomarkers, namely SRY, DAZ2 and TSPY1 genes and the internal control, ACTB gene. The multiplex PCR disclosed herein detected DNA from various sources including, importantly, cffDNA which is present in material plasma, serum and urine from the 9^{th} week fetal embryo. This multiplex PCR is an appropriate methodology for determining sex from all source of fetal DNA including highly degraded cffDNA. It may be performed using nucleated, fetal red blood cells (CD71⁺) or cord blood as a DNA source. Paternal and maternal samples, for example, from buccal cells, may be used as controls. These properties lower the expense of gender determination and increase its accuracy due to the use of three independent Y chromosome markers. The multiplex PCR can be performed noninvasively using cffDNA from a blood sample.

Male gender is preferably determined by detecting the presence of two out of three, or three out of three Y chromosome markers or a single Y marker with ≥1,000,000 intensity.

The Y chromosome based markers using the presently developed multiplex PCR can be a reliable methodology for the determination of fetal sex using invasive and non-invasive source of fetal DNA, which cannot be done in the first trimester by means of ultrasonography due to the incomplete external genitalia. The shortest possible time is sufficient to determine using single tube multiplex PCR with 3 Y markers. The study confirms the gender determination procedure using male-bearing pregnancies. cffDNA from invasive, non-invasive and 9^{th} week embryo were used as a resource for the determination of sex using highly sensitive and specific multiplex PCR.

1. A multiplex method for simultaneous detection of Y chromosome-specific target DNA of the SRY, DAZ2 and TSPY1 genes and control DNA of the ACTB gene in a sample containing DNA, comprising:
(a) simultaneously amplifying portions of the SRY, DAZ2, TSPY1 and ACTB genes by a polymerase chain reaction (PCR) to produce four different amplicons; wherein each amplicon has a different distinguishable length;
(b) detecting the presence or absence of amplicons from the SRY, DAZ2 and TSPY1 and ACTB and determining the length of each amplicon when present,
selecting a sample containing Y chromosome DNA when presence of amplicons for at least two of SRY, DAZ2 or TSPY1 is detected, or
selecting a sample containing Y chromosome DNA when presence of one or more amplicons from SRY, DAZ2 or TSPY1 having an intensity of ≥1,000,000 intensity is detected; or
selecting a sample not containing Y chromosome DNA when no amplicons from at least two of SRY, DAZ2 or TSPY1 are detected or when only a presence of a single amplicon from SRY, DAZ2 or TSPY1 having an intensity lower than 1,000,000 is detected; and, optionally,
sequencing the amplicons to confirm their origination from segments of SRY, DAZ2, TSPY1 and/or ACTB.

2. The method of claim 1, wherein the sample comprises previously frozen, dried, desiccated, stored, or degraded DNA.

3. The method of claim 1, wherein the sample comprises fetal DNA.

4. The method of claim 1, wherein the sample comprises maternal cell-free plasma or maternal cell-free serum from a pregnant woman.

5. The method of claim 1, wherein the sample comprises peripheral blood monocytes that are CD71 positive.

6. The method of claim 1, wherein the sample is urine.

7. The method of claim 1, wherein the sample is cord blood, amniotic fluid, fetal cells, fetal tissue or fetal blood.

8. The method of claim 1, further comprising isolating DNA from a mother's buccal cells and/or from a father's blood or buccal cells; and
(a) simultaneously amplifying portions of the SRY, DAZ2, TSPY1 and ACTB to produce amplicons; wherein each amplicon has a different distinguishable length; and
(b) detecting the presence or absence of amplicons from the SRY, DAZ2 and TSPY1 and ACTB in the maternal or paternal DNA by determining the length of each amplicon.

9. The method of claim 1, further comprising isolating the target DNA from the cellular, protein, and other components of the biological sample.

10. The method of claim 1, further comprising isolating fetal cells expressing CD71 from cells that do not express CD71, and extracting fetal DNA from said isolated cells, wherein the isolated DNA constitutes the sample.

11. The method of claim 1, further comprising contacting a sample with sequence-specific DNA probes to capture segments of SRY, DAZ2 or TSPY1 or ACTB genes for amplification.

12. The method of claim 1, wherein the polymerase chain reaction amplifies the DNA using four sets of primers which comprise:
a first primer comprising SEQ ID NO: 1 and a second primer comprising SEQ ID NO: 2 which amplify the SRY target nucleic acid sequence;
a third primer comprising SEQ ID NO: 7 and a fourth primer comprising SEQ ID NO:8 which amplify the DAZ2 target nucleic acid sequence;
a fifth primer comprising SEQ ID NO: 5 and a sixth primer comprising SEQ ID NO:6 which amplify the TSPY1 target nucleic acid sequence; and
a seventh primer comprising SEQ ID NO: 3 and an eighth primer consisting of SEQ ID NO: 4 which amplify the ACTB target nucleic acid sequence.

13. The method of claim 1, wherein the polymerase chain reaction amplifies the DNA using four sets of primers which comprise:
a first primer comprising SEQ ID NO: 1 and a second primer comprising SEQ ID NO: 2 which amplify the SRY target nucleic acid sequence;
a third primer comprising SEQ ID NO: 7 and a fourth primer comprising SEQ ID NO: 8 which amplify the DAZ2 target nucleic acid sequence;
a fifth primer comprising SEQ ID NO: 5 and a sixth primer comprising SEQ ID NO: 6 which amplify the TSPY1 target nucleic acid sequence; and a seventh primer comprising SEQ ID NO: 3 and
an eighth primer consisting of SEQ ID NO: 4 which amplify the ACTB target nucleic acid sequence;
wherein said primers comprise one or more modified nucleotides selected from the group consisting of 2′-O-methyl nucleotides, 2′-fluoro-nucleotides, 2′-amino nucleotides, and arabinose nucleotides.

14. The method of claim 1, wherein the sample is a fetal sample and one or both parents have or are carriers of an X-linked genetic disease.

15. The method of claim 1, wherein the sample is a fetal sample, and wherein said method further comprises determining the karyotype of the fetus.

16. The method of claim 1, wherein the sample is a fetal sample, and wherein said method further comprises testing the fetus for muscular dystrophy, fragile X syndrome, hemophilia, or for X-linked lymphoproliferative syndrome.

17. The method of claim 1, wherein the sample is a fetal sample, and wherein said method further comprises treating a male fetus for an X-linked genetic disease.

18. The method of claim 1, wherein the sample is a fetal sample, and wherein said method further comprises treating a female fetus for an X-linked genetic disease.

19. A kit for determining gender of a biological sample comprising a set of primers that amplify segments of SRY, DAZ2, TSPY1 and ACTB genes to produce amplicons of different lengths; and, optionally one or more reagents required for PCR, reaction containers, packaging materials, and instructions.

20. The kit of claim 19 that comprises a first primer comprising SEQ ID NO:1 and a second primer comprising SEQ ID NO:2 which amplify the SRY target nucleic acid sequence;
a third primer comprising SEQ ID NO: 7 and a fourth primer comprising SEQ ID NO:8 which amplify the DAZ2 target nucleic acid sequence;
a fifth primer comprising SEQ ID NO:5 and a sixth primer comprising SEQ ID NO:6 which amplify the TSPY1 target nucleic acid sequence; and a seventh primer comprising SEQ ID NO:3 and
an eighth primer consisting of SEQ ID NO: 4 which amplify the ACTB target nucleic acid sequence; and optionally,
wherein one or more of said primers comprise one or more modified nucleotides selected from the group consisting of 2′-O-methyl nucleotides, 2′-fluoro-nucleotides, 2′-amino nucleotides, and arabinose nucleotides.


SEQUENCE LISTING (12 sequences)

SEQ ID NO: 1 - DNA, 34 bp, synthetic construct
  misc_feature 1..34: note = Primer name SRYaF; gene name SRY
  source 1..34: mol_type = other DNA
tgttgagggcggagaaatgcaagtttcattacaa

SEQ ID NO: 2 - DNA, 35 bp, synthetic construct
  misc_feature 1..35: note = Primer name SRYaR; gene name: SRY
  source 1..35: mol_type = other DNA
atgttacccgattgtcctacagctttgtccagtgg

SEQ ID NO: 3 - DNA, 25 bp, synthetic construct
  misc_feature 1..25: note = Primer name ActinF; gene name: ACTB
  source 1..25: mol_type = other DNA
actttctgcatgtcccccgtctggc

SEQ ID NO: 4 - DNA, 24 bp, synthetic construct
  misc_feature 1..24: note = Primer name ActinR; gene name ACTB
  source 1..24: mol_type = other DNA
gccgggagacagtctccactcacc

SEQ ID NO: 5 - DNA, 21 bp, synthetic construct
  misc_feature 1..21: note = Primer name DYS14F; gene name TSPY1
  source 1..21: mol_type = other DNA
ggtgccagagaggctgcggca

SEQ ID NO: 6 - DNA, 24 bp, synthetic construct
  misc_feature 1..24: note = Primer name:DYS14R; gene name:TSPY1
  source 1..24: mol_type = other DNA
taaccggctccagctccacctgaa

SEQ ID NO: 7 - DNA, 39 bp, synthetic construct
  misc_feature 1..39: note = Primer name:DAZ2aF; Gene name: DAZ2
  source 1..39: mol_type = other DNA
gaaaatttgtagaacagagacagaaatgctttgctgtta

SEQ ID NO: 8 - DNA, 36 bp, synthetic construct
  misc_feature 1..36: note = Primer name DAZ2aF; gene name DAZ2
  source 1..36: mol_type = other DNA
cccgaaccagaatatatccagaagtcagcaatttat

SEQ ID NO: 9 - DNA, 700 bp, Homo sapiens
  misc_feature 1..700: note = SRY
  source 1..700: mol_type = other DNA
tctggtagaagtgagttttggatagtaaaataagtttcgaactctggcacctttcaattttgtcgcactctccttgtttttgacaatgcaatcatatgcttctgctatgttaagcgtattcaacagcgatgattacagtccagctgtgcaagagaatattcccgctctccggagaagctcttccttcctttgcactgaaagctgtaactctaagtatcagtgtgaaacgggagaaaacagtaaaggcaacgtccaggatagagtgaagcgacccatgaacgcattcatcgtgtggtctcgcgatcagaggcgcaagatggctctagagaatcccagaatgcgaaactcagagatcagcaagcagctgggataccagtggaaaatgcttactgaagccgaaaaatggccattcttccaggaggcacagaaattacaggccatgcacagagagaaatacccgaattataagtatcgacctcgtcggaaggcgaagatgctgccgaagaattgcagtttgcttcccgcagatcccgcttcggtactctgcagcgaagtgcaactggacaacaggttgtacagggatgactgtacgaaagccacacactcaagaatggagcaccagctaggccacttaccgcccatcaacgcagccagctcaccgcagcaacgggaccgctacagccactggacaaagctgtag

SEQ ID NO: 10 - DNA, 288 bp, Homo sapiens
  misc_feature 1..288: note = DYS14
  source 1..288: mol_type = other DNA
ggccttggtgtgtgcgagtgccaaggagggcaccgccttcaggatggaggctgtrcaggagggggcggccggggtggagagtgagcaggcggctttgggggaggaggcggtgctgctgttggatgacataatggcggaggtggaggtggtggcggaggaggagggcctcgtggagsggcgggaggaggcccagcgggcacagcaggctgtgcctggccctgggcccatgaccccagagtctgcactggaggagctgctggccgttcaggtggagctggagccggttaa

SEQ ID NO: 11 - DNA, 215 bp, Homo sapiens
  source 1..215: mol_type = other DNA
aataggagtaaacgagagtattacctccaaagcaccagagctgctttcctccttataaccagttcctaaagtgaatgaaagcagctctccttatgtgtctgcctacttcattcttcggtaagtttaacagttcatctagctaccctttatttgaaatgatttccagatgcctcctcatataaattgctgacttctggatatattctggttcggga

SEQ ID NO: 12 - DNA, 476 bp, Homo sapiens
  misc_feature 1..476: note = Actin
  source 1..476: mol_type = other DNA
tctctgccttacagatcatgtttgagaccttcaacaccccagccatgtacgttgctatccaggctgtgctatccctgtacgcctctggccgtaccactggcatcgtgatggactccggtgacggggtcacccacactgtgcccatctacgaggggtatgccctcccccatgccatcctgcgtctggacctggctggccgggacctgactgactacctcatgaagatcctcaccgagcgcggctacagcttcaccaccacggccgagcgggaaatcgtgcgtgacattaaggagaagctgtgctacgtcgccctggacttcgagcaagagatggccacggctgcttccagctcctccctggagaagagctacgagctgcctgacggccaggtcatcaccattggcaatgagcggttccgctgccctgaggcactcttccagccttccttcctgggtgagtggagactgtctcccggc